(19) United States
(12) Patent Application Publication
(10) Pub. No.: US 2008/0081022 A1
(43) Pub. Date: Apr. 3, 2008

(54) Poss Containing Cosmetic Compositions Having Improved Wear And/Or Pliability And Methods Of Making Improved Cosmetic Compositions

(75) Inventors: Weih Hong Yu, Edison, NJ (US); Murat Quadir, Scotch Plains, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/663,110

(22) PCT Filed: Sep. 12, 2005
(86) PCT No.: PCT/US05/32502

§ 371 Date: Mar. 13, 2007

Related U.S. Application Data
(60) Provisional application No. 60/609,482, filed on Sep. 13, 2004.

(51) Int. Cl. A61K 8/89; A61K 8/02; A61Q 1/02; A61Q 1/04; A61Q 1/08; A61Q 1/10; A61Q 3/02

(52) U.S. Cl. 424/43; 424/61; 424/63; 424/64; 424/69; 424/70.7; 424/78.37

(57) ABSTRACT

Film-forming and cosmetic compositions containing a combination of certain polyhedral oligomeric silsesquioxanes (POSS) and non-POSS silicone resins are described, the combination providing improved wear and/or pliability properties over previous silicone resin containing compositions. Typically, the advantageous combinations will be formulated in a cosmetic composition that contains at least one solvent, and, preferably, a colorant, such as a pigment. Other cosmetic ingredients known to those of skill in the art can also be combined therewith, depending on the desired product, such as a foundation, mascara, lip coating (lip stick, lip gloss, lip pencil and the like), nail coating and the like. Methods of making cosmetic compositions including combinations of a POSS and non-POSS silicone resin providing the advantageous properties are also described.

### CROSS-REFERENCE TO RELATED APPLICATIONS

This application claims the benefit of the filing date of U.S. Provisional Patent Application No. 60/609,482 filed Sep. 13, 2004, the disclosure of which is hereby incorporated herein by reference.

### BACKGROUND OF THE INVENTION

The design of cosmetics, such as foundations, concealers, blushes, mascaras, nail coatings, lip coatings, eyeliners, and eye shadows, is a complex process which involves, among other things, consideration of the substrate to which the cosmetic is to be applied, the method of application and, generally, formulating the composition to achieve any one of the desirable properties known to those of skill in the cosmetics industry. For example, one or more properties such as appearance, viscosity, workability, brittleness, feel, wear, UV protection, anti-transferability, breathability, ease of removal, and the like may need to be taken into consideration.

Of these properties, “wear,” which relates to how well a cosmetic retains its color, coverage, texture and/or consistency over time, has been the subject of much research and development. With respect to film-forming cosmetics, another beneficial property is “pliability,” a property which relates to the brittleness of the film, which can impact wear, appearance, texture and feel. In the cosmetics industry there is still a need for formulations which improve both of these characteristics, wear and pliability.

One class of compounds which has been the subject of much research as a possible way of improving upon many of the desired properties of cosmetic compositions are the silicone resins. “Silicone resins” refers to a variety of polymers which are characterized by repeating Si subunits having at least one and up to four oxygen bridges with other Si atoms. Of the four possible Si bonds, instead of oxygen bridges, up to three R-groups can be present. By varying the subunits and substituents, a vast variety of polymers can be created.

Silicone resins have been disclosed previously in connection with cosmetic formulations with allegedly improved wear and/or pliability. For example, U.S. Patent Application Publication No. US2002/0114773A1, which published on Aug. 22, 2002, to Kanji et al., discloses, in one embodiment, transfer resistant cosmetic compositions, which may also be pliable and comfortable to wear upon application to a substrate. The compositions comprise, more particularly, at least one structuring polymer and at least one silicone resin. Among the silicone resins disclosed, for example, are polymethyl silsesquioxanes being formed primarily of polymerized repeating subunits of CH₃SiO_{3/2}. See also U.S. Patent Application Publication No. US2002/0031488A1, EP 0,624,594 and U.S. Pat. Nos. 2,465,188, 5,047,492, 5,246,694 and 5,439,673.

It is also believed that silicone resins substituted with propyl and phenyl groups have been sold by Wacker under the trademark BELSIL® silicones SPR 45 and used in connection with a lipstick and a hand protection lotion. BELSIL® silicones SPR 45 is a T resin (formed predominantly of Si subunits having three oxygen bridges), has a random structure, and is not believed to be in a cage formation such as the POSS compounds discussed in the following paragraphs.

Another class of silicone resins are the Polyhedral Oligomeric Silsesquioxanes or “POSS”. These compounds are distinguished from other silicone resins by their rigid three-dimensional cage-like structures.

One POSS-containing silicone resin known as BELSIL® PMS MK, Siliconharz MK, or, more commonly, Resin MK has previously been disclosed in Kanji et al. in connection with cosmetic formulations. Resin MK is believed to contain a mixture of silicone compounds which are in a “ladder” (non POSS three-dimensional structure) and cage (POSS) configuration of 8 Si subunits entirely substituted with methyl groups. It is further believed that these mixtures include a majority of molecules in the ladder, or non-POSS, configuration.

POSS has also previously been described in, for example, U.S. Published Patent Application No. 2004/0120915 to Yang et al., which is incorporated by reference with respect to its disclosure of a variety of POSS molecules. In addition, POSS is described in the commonly owned U.S. patent application Ser. Nos. 10/799,281, entitled “POSS CONTAINING COSMETICS AND PERSONAL CARE PRODUCTS,” filed Mar. 12, 2004, and 10/799,280, entitled “EPOSS CONTAINING COSMETICS AND PERSONAL CARE PRODUCTS,” filed Mar. 12, 2004, which are incorporated herein by reference with respect to their disclosures relating to POSS and cosmetic formulations. See also U.S. Pat. Nos. 6,127,557; 5,858,544; and 2,465,188. Numerous molecules are sold by Hybrid Plastics.

### SUMMARY OF THE INVENTION

The present invention advances the science of cosmetics formulation by providing certain combinations of POSS and non-POSS silicone resins, which, when employed in a film forming or cosmetic composition, have improved wear and/or pliability over previously known silicone-resin containing compositions. Those skilled in the art will be able to use these advantageous combinations to create, for example, foundations, mascaras, eye liners, concealers, lip coatings, blushes, body make-up, nail coatings, and other cosmetics that are longer lasting after application, have better appearance, texture and feel over time, and/or are more easily processed than previously disclosed silicone-resin containing compositions.

In one aspect of the present invention, a film forming composition is provided containing a combination of a POSS and non-POSS silicone resin, wherein the non-POSS silicone resin is either a siloxysilicate or a polysiloxane. Siloxysilicates have the formula [R₃—Si—O]x-(SiO_{4/2})y, wherein x and y range from about 50 to about 80, and polysiloxanes have the formula [R₃—Si—O]—(R₂SiO)_{X}—[Si—R₃], wherein X is at least 2000. The R groups can be, for example, an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin. Preferably, the POSS is an iso-octyl POSS, and the polysiloxane is dimethicone.

In particular, it has been found that the advantageous properties described above can be obtained by combining the POSS and non-POSS resins in particular amounts. Generally, there will be more non-POSS resin than POSS. For example, the ratio of POSS to the non-POSS silicone resin can range from about 1:4 to about 1:1, more particularly, from about 1:3 to about 9:11, and even more particularly, from about 3:7 to about 2:3.

In the combination with a siloxysilicate or polysiloxane, POSS will have the characteristic three-dimensional cage structure and will have at least one M, D or T subunit, as described in further detail below. M, D and T subunits have at least one R group, which can be, for example, a hydrogen, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻N⁺(CH₃)₃, —OH, —(CH₂)ₙN⁺H₃X⁻ wherein n is 0-30 and X is a counter ion,

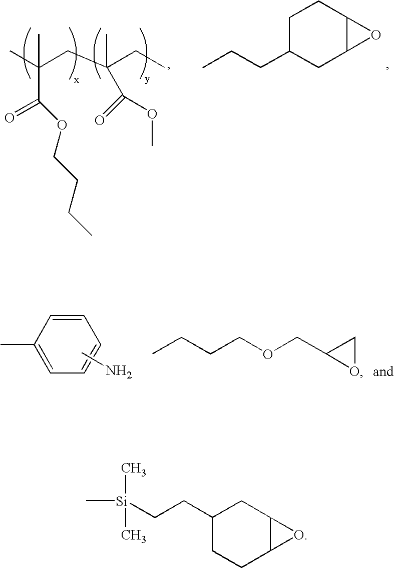

In another embodiment of the invention, a film forming composition is provided including a combination of a POSS having a three dimensional cage structure formed of a plurality of Si subunits, at least one of the subunits having one or more R groups, and a non-POSS silicone resin. In this embodiment, if the silicone resin is a polymethylsilsesquioxane, then the POSS will have other than 8 Si subunits, or at least one of the R groups of the POSS will be other than a methyl group, or the POSS will have an incomplete cage structure.

As in the previous embodiment, the ratio of POSS to the non-POSS silicone resin will range from about 1:4 to about 1:1, more particularly, from about 1:3 to about 9:11, and even more particularly, from about 3:7 to about 2:3.

In addition to film-forming compositions, cosmetic compositions are also provided. In one embodiment a cosmetic composition is provided which includes a combination of a POSS having a three dimensional cage structure formed of a plurality of Si subunits, at least one of the subunits having one or more R groups, and a non-POSS silicone resin. In these embodiments, if the silicone resin is a polymethylsilsesquioxane, then the POSS will have other than 8 Si subunits, or at least one of the R groups of the POSS will something other than a methyl group, or the POSS will have an incomplete cage structure. As in many conventional cosmetic compositions, the composition can include a solvent, a coloring agent, and, optionally, one or more additional cosmetic ingredients. Typically, the combined amount of POSS and non-POSS silicone resin will be in the range of about 0.5% to about 35% by weight of the total cosmetic composition. Again, the ratio of POSS to the non-POSS silicone resin will range from about 1:4 to about 1:1, more particularly, from about 1:3 to about 9:11, and even more particularly, from about 3:7 to about 2:3.

In a preferred cosmetic composition, the non-POSS silicone resin is a Q resin. A preferred POSS for use in connection with cosmetic compositions is an iso-octyl POSS. The cosmetic compositions can include, for example, foundations, mascaras, eye liners, concealers, lip coatings, blushes, body make-up, and nail coatings. In producing these cosmetics, additional cosmetic ingredients are typically employed. For example, the cosmetic composition can include one or more cosmetic ingredients, for example, an absorbent, alphahydroxy acid, betahydroxy acid, polyhydroxy acid, antiacne agent, anticaking agent, antifoaming agent, antimicrobial agent, antioxidant, astringent, binder, bleaching agent, buffer, biological additive, bulking agent, carrier, chelating agent, coupling agent, compatibilizer, conditioner, colorant, cosmetic astringent, cosmetic biocide, denaturant, drug astringent, detergent, deodorant, dispersant, external analgesic, emulsifier, film former, foaming agent, fragrance and fragrance component, humectant, keratolytic, moisturizer, straightening agent, oxidizer, mineral and organic particle, plastic, polymer, opacifying agent, perfume, pH adjuster, preservative, protein, retinoid, reducing agent, sequesterant, skin bleaching agent, skin conditioning agent, skin smoothing agent, skin soothing agent, skin healing agent, softener, solubilizing agent, surfactant, lubricant, thickener, penetrant, permeation enhancer, analgesic, anti-inflammatory agent, antibiotic, anesthetic, plasticizer, salt, solvent essential oil, sunscreen and UV-absorber, vitamin, provitamine, plant extract, ceramide and pseudoceramide. The cosmetic compositions can be formulated, for example, as a liquid, solid, cream, ointment, solution, gel, mousse, stick, spray, powder, emulsion or dispersion.

In another embodiment, methods of making a cosmetic composition having one or more of the improved properties are also provided. The process comprises predetermining the amount of POSS and non-POSS silicone resin necessary to impart improved wear or flexibility to the composition, the POSS having a three dimensional cage structure formed of a plurality of Si subunits, at least one of the Si subunits having one or more R groups, wherein, if the non-POSS silicone resin comprises a polymethylsilsesquioxane, then the POSS comprises other than 8 Si subunits, or at least one of the R groups of the POSS is other than a methyl group, or the POSS has an incomplete cage structure; and combining the POSS and non-POSS silicone resin with another cosmetic ingredient.

In another embodiment, the process of predetermining the amounts of POSS and non-POSS resin includes preparing a film forming composition from the POSS and non-POSS resins, applying the film forming composition to a substrate to form a film; and testing the brittleness of the film.

Additional embodiments of the present invention are as follows:

In one instance, a film forming composition comprises at least one POSS and at least one non-POSS silicone resin, wherein the non-POSS silicone resin is a siloxysilicate.

In yet another instance, the siloxysilicate is of the formula [R₃—Si—O]ₓ—(SiO_{4/2})_{y}, wherein x and y range from about 50 to about 80, and R is an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin.

In another instance, the siloxysilicate is a trimethylsiloxysilicate.

In another instance the POSS comprises a plurality of subunits forming a three-dimensional cage structure, at least one of said subunits being an M, D or T subunit having at least one R group.

In another instance, the R group is a hydrogen, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻N⁺(CH₃)₃, —OH, —(CH₂)ₙN⁺H₃X⁻ wherein n is 0-30 and X is a counter ion,

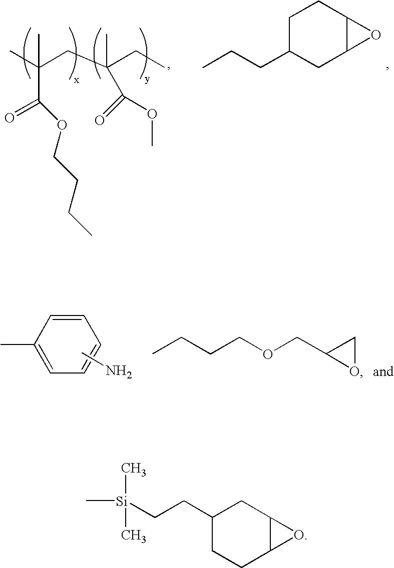

In another instance, the at least one POSS is an Isooctyl-POSS.

In another instance, the ratio of the POSS to said non-POSS silicone resin comprises from about 1:4 to about 1:1.

In another instance, the ratio of the POSS to said non-POSS silicone resin comprises from about 1:3 to about 9:11.

In another instance, the ratio of the POSS to said silicone resin comprises from about 3:7 to about 2:3.

In another instance, the POSS is a liquid at room temperature.

In yet another instance, a film forming composition comprises at least one POSS and at least one non-POSS silicone resin, wherein said non-POSS silicone resin is a polysiloxane.

In another instance, the polysiloxane is of the formula [R₃—Si—O]—(R₂SiO)_{X}—[Si—R₃], wherein X is at least 2000, and R is an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin.

In another instance, the polysiloxane is dimethicone.

In another instance, the POSS comprises a plurality of subunits forming a three-dimensional cage structure, at least one of said subunits being an M, D or T subunit having at least one R group.

In another instance, the R group is a hydrogen, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻N⁺(CH₃)₃, —OH, —(CH₂)ₙN⁺H₃X⁻ wherein n is 0-30 and X is a counter ion,

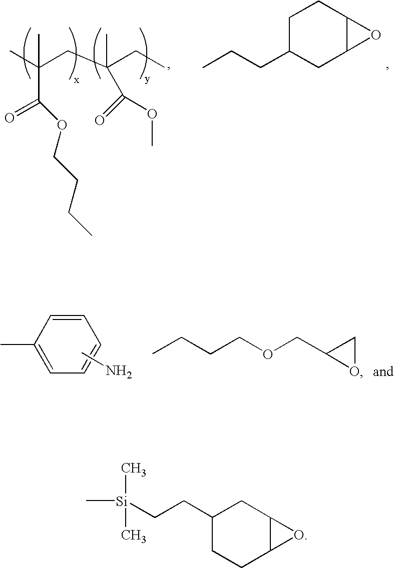

In another instance, the at least one POSS is an Isooctyl-POSS.

In another instance, the ratio of the POSS to said non-POSS silicone resin comprises from about 1:4 to about 1:1.

In another instance, the ratio of the POSS to said non-POSS silicone resin comprises from about 1:3 to about 9:11.

In another instance, the ratio of the POSS to said silicone resin comprises from about 3:7 to about 2:3.

In another instance, the POSS is a liquid at room temperature.

In yet another instance, film forming composition comprises at least one POSS having a three-dimensional cage structure formed of a plurality of Si subunits, at least one of said Si subunits having one or more R groups; at least one silicone resin other than a POSS wherein if the silicone resin comprises a polymethylsilsesquioxane, then the POSS comprises other than 8 Si subunits, or at least one of the R groups of said POSS is other than a methyl group, or said POSS has the structure of an incomplete cage.

In another instance, the ratio of the POSS to said silicone resin comprises from 1:4 to about 1:1.

In another instance, the ratio of the POSS to said silicone resin comprises from about 1:3 to about 9:11.

In another instance, the ratio of the POSS to said silicone resin comprises from about 3:7 to about 2:3.

In another instance, the POSS is a liquid at room temperature.

In yet another instance, a cosmetic composition comprises at least one POSS, said POSS comprising at least 6 Si subunits forming a three-dimensional cage structure; at least one non-POSS silicone resin comprising at least two Si atoms, said Si atoms being bound to at least one other of said Si atoms by at least one oxygen bridge, at least one of said Si atoms having one or more R groups other than a methyl group; a solvent; and a coloring agent.

In another instance, the non-POSS silicone resin is an MQ resin.

In another instance, the R group is an alkyl other than methyl, alcohol, alkoxysilane, amine, chlorosilane, epoxide, ester, halide, methacrylate, molecular silica, nitrile, norborene, olefin, phosphine, silane, silanol, styrenic polymer, or polyolefin.

In another instance, the silicone resin has a plurality of R groups other than a methyl, said R groups being the same or different, wherein said R groups are an alkyl other than methyl, alcohol, alkoxysilane, amine, chlorosilane, epoxide, ester, halide, methacrylate, molecular silica, nitrile, norborene, olefin, phosphine, silane, silanol, styrenic polymer, or polyolefin.

In another instance, the total amount of the POSS and silicone resin comprises from about 0.5% to about 35% by weight of said cosmetic composition.

In another instance, the ratio of the POSS to said silicone resin comprises from about 1:4 to about 1:1.

In another instance, the ratio of the POSS to said silicone resin comprises from about 1:3 to about 9:11.

In another instance, the ratio of the POSS to said silicone resin comprises from about 3:7 to about 2:3.

In another instance, the composition further comprises one or more cosmetic ingredients.

In yet another instance, the cosmetic ingredient comprises an absorbent, alphahydroxy acid, betahydroxy acid, polyhydroxy acid, antiacne agent, anticaking agent, antifoaming agent, antimicrobial agent, antioxidant, astringent, binder, bleaching agent, buffer, biological additive, bulking agent, carrier, chelating agent, coupling agent, compatibilizer, conditioner, colorant, cosmetic astringent, cosmetic biocide, denaturant, drug astringent, detergent, deodorant, dispersant, external analgesic, emulsifier, film former, foaming agent, fragrance and fragrance component, humectant, keratolytic, moisturizer, straightening agent, oxidizer, mineral and organic particle, plastic, polymer, opacifying agent, perfume, pH adjuster, preservative, protein, retinoid, reducing agent, sequesterant, skin bleaching agent, skin conditioning agent, skin smoothing agent, skin soothing agent, skin healing agent, softener, solubilizing agent, surfactant, lubricant, thickener, penetrant, permeation enhancer, analgesic, anti-inflammatory agent, antibiotic, anesthetic, plasticizer, salt, solvent essential oil, sunscreen and UV-absorber, vitamin, provitamine, plant extract, ceramide and pseudoceramide.

In another instance, the cosmetic composition comprises a foundation, mascara, eye liner, concealer, lip coating, blush for cheeks or eyelids, body makeup or nail coating.

In another instance, the cosmetic composition is formulated in the form of a liquid, solid, cream, ointment, solution, gel, mousse, stick, spray, powder, emulsion or dispersion.

In yet another instance, a cosmetic composition comprises at least one POSS, said POSS comprising other than 8 Si subunits forming a three-dimensional cage structure; at least one silicone resin other than a POSS; a solvent; and a coloring agent.

In another instance, the POSS comprises more than 8 Si subunits.

In another instance, the POSS comprises 6 or 7 Si subunits.

In another instance, the at least one of the subunits is an M, D or T subunit having at least one R group.

In another instance, the R group is a hydrogen, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻N⁺(CH₃)₃, —OH, —(CH₂)ₙN⁺H₃X⁻ wherein n is 0-30 and X is a counter ion,

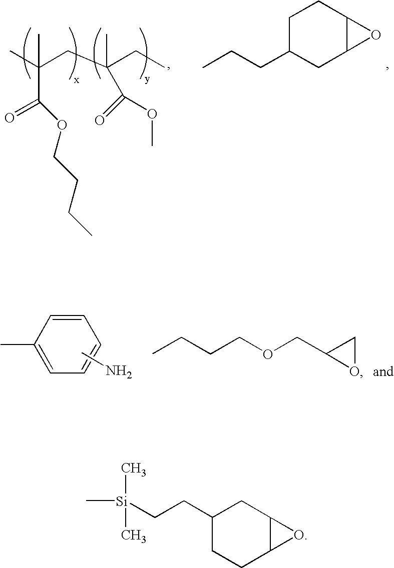

In another instance, the non-POSS silicone resin is an MQ resin.

In another instance, the at least one silicone resin comprises at least two Si atoms, said Si atoms being bound to at least one other of said Si atoms of said silicone resin by at least one oxygen bridge, at least one of said Si atoms having one or more R groups.

In another instance, the R group of said silicone resin is an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin.

In another instance, the at least one silicone resin is trimethylsyloxysilicate.

In another instance, the total amount of the POSS and silicone resin comprises from about 0.5% to about 35% by weight of said cosmetic composition.

In another instance, the ratio of the POSS to said silicone resin comprises from about 1:4 to about 1:1.

In another instance, the ratio of the POSS to said silicone resin is from about 1:3 to about 9:11.

In another instance, the ratio of the POSS to said silicone resin comprises from about 3:7 to about 2:3.

In another instance, the composition further comprises one or more cosmetic ingredients.

In another instance, the cosmetic ingredient comprises an absorbent, alphahydroxy acid, betahydroxy acid, polyhydroxy acid, antiacne agent, anticaking agent, antifoaming agent, antimicrobial agent, antioxidant, astringent, binder, bleaching agent, buffer, biological additive, bulking agent, carrier, chelating agent, coupling agent, compatibilizer, conditioner, colorant, cosmetic astringent, cosmetic biocide, denaturant, drug astringent, detergent, deodorant, dispersant, external analgesic, emulsifier, film former, foaming agent, fragrance and fragrance component, humectant, keratolytic, moisturizer, straightening agent, oxidizer, mineral and organic particle, plastic, polymer, opacifying agent, perfume, pH adjuster, preservative, protein, retinoid, reducing agent, sequesterant, skin bleaching agent, skin conditioning agent, skin smoothing agent, skin soothing agent, skin healing agent, softener, solubilizing agent, surfactant, lubricant, thickener, penetrant, permeation enhancer, analgesic, anti-inflammatory agent, antibiotic, anesthetic, plasticizer, salt, solvent essential oil, sunscreen and UV-absorber, vitamin, provitamine, plant extract, ceramide and pseudoceramide.

In another instance, the cosmetic composition comprises a foundation, mascara, eye liner, concealer, lip coating, blush for cheeks or eyelids, body makeup or nail coating.

In another instance, the cosmetic composition is formulated in the form of a liquid, solid, cream, ointment, solution, gel, mousse, stick, spray, powder, emulsion or dispersion.

In yet another instance, a cosmetic composition comprises at least one POSS having a three dimensional cage structure, said POSS comprising 6 or more Si subunits, at least one of said Si subunits having an R group which is other than a methyl group; a silicone resin other than a POSS; and a colorant.

In another instance, the R group is a hydrogen, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻N⁺(CH₃)₃, —OH, —(CH₂)ₙN⁺H₃X⁻ wherein n is 0-30 and X is a counter ion,

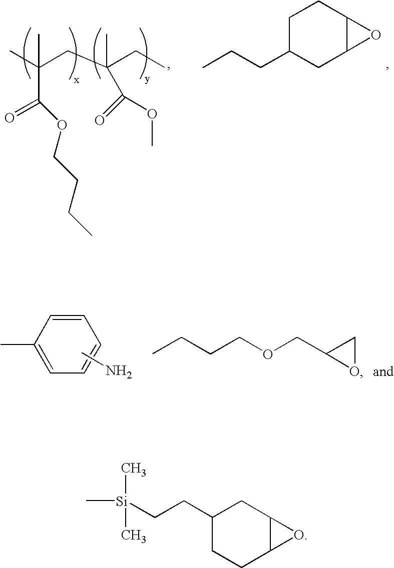

In another instance, the POSS is an Isooctyl-POSS.

In another instance, the at least one silicone resin comprises at least two Si atoms, said Si atoms being bound to at least one other of said Si atoms of said silicone resin by at least one oxygen bridge, at least one of said Si atoms having one or more R groups.

In another instance, the R group of said silicone resin is an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin.

In another instance, the non-POSS silicone resin is an MQ resin.

In another instance, the at least one silicone resin is trimethylsyloxysilicate.

In another instance, the total amount of the POSS and silicone resin comprises from about 0.5% to about 35% by weight of said cosmetic composition.

In another instance, the ratio of the POSS to said silicone resin comprises from about 1:4 to about 1:1.

In another instance, the ratio of the POSS to said silicone resin comprises from about 1:3 to about 9:11.

In another instance, the ratio of the POSS to said silicone resin comprises from about 3:7 to about 2:3.

In another instance, the composition further comprises one or more cosmetic ingredients.

In another instance, the cosmetic ingredient comprises an absorbent, alphahydroxy acid, betahydroxy acid, polyhydroxy acid, antiacne agent, anticaking agent, antifoaming agent, antimicrobial agent, antioxidant, astringent, binder, bleaching agent, buffer, biological additive, bulking agent, carrier, chelating agent, coupling agent, compatibilizer, conditioner, colorant, cosmetic astringent, cosmetic biocide, denaturant, drug astringent, detergent, deodorant, dispersant, external analgesic, emulsifier, film former, foaming agent, fragrance and fragrance component, humectant, keratolytic, moisturizer, straightening agent, oxidizer, mineral and organic particle, plastic, polymer, opacifying agent, perfume, pH adjuster, preservative, protein, retinoid, reducing agent, sequesterant, skin bleaching agent, skin conditioning agent, skin smoothing agent, skin soothing agent, skin healing agent, softener, solubilizing agent, surfactant, lubricant, thickener, penetrant, permeation enhancer, analgesic, anti-inflammatory agent, antibiotic, anesthetic, plasticizer, salt, solvent essential oil, sunscreen and UV-absorber, vitamin, provitamine, plant extract, ceramide and pseudoceramide.

In another instance, the cosmetic composition comprises a foundation, mascara, eye liner, concealer, lip coating, blush for cheeks or eyelids, body makeup or nail coating.

In another instance, the cosmetic composition is formulated in the form of a liquid, solid, cream, ointment, solution, gel, mousse, stick, spray, powder, emulsion or dispersion.

In yet another instance, a cosmetic composition comprises at least one POSS comprising an incomplete three dimensional cage structure formed of 6 or more Si subunits; a silicone resin other than a POSS; a solvent; and a colorant.

In another instance, at least one of the subunits has at least one R group, said R group being a hydrogen, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻N⁺(CH₃)₃, —OH, —(CH₂)ₙN⁺H₃X⁻ wherein n is 0-30 and X is a counter ion,

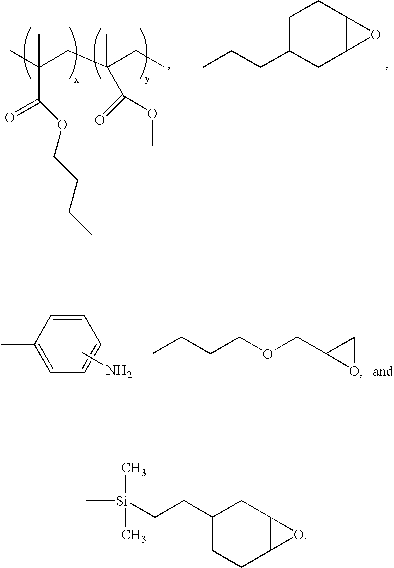

In another instance, at least one silicone resin comprises at least two Si atoms, said Si atoms being bound to at least one other of said Si atoms of said silicone resin by at least one oxygen bridge, at least one of said Si atoms having one or more R groups.

In another instance, the R group of said silicone resin is an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin.

In another instance, the non-POSS silicone resin is an MQ resin.

In another instance, the at least one silicone resin is trimethylsyloxysilicate.

In another instance, the total amount of said POSS and silicone resin comprises from about 0.5% to about 35% by weight of said cosmetic composition.

In another instance, the ratio of said POSS to said silicone resin comprises from about 1:4 to about 1:1.

In another instance, the ratio of said POSS to said silicone resin comprises from about 1:3 to about 9:11.

In another instance, the ratio of said POSS to said silicone resin comprises from about 3:7 to about 2:3.

In another instance, the composition further comprises one or more cosmetic ingredients.

In another instance, the cosmetic ingredient comprises an absorbent, alphahydroxy acid, betahydroxy acid, polyhydroxy acid, antiacne agent, anticaking agent, antifoaming agent, antimicrobial agent, antioxidant, astringent, binder, bleaching agent, buffer, biological additive, bulking agent, carrier, chelating agent, coupling agent, compatibilizer, conditioner, colorant, cosmetic astringent, cosmetic biocide, denaturant, drug astringent, detergent, deodorant, dispersant, external analgesic, emulsifier, film former, foaming agent, fragrance and fragrance component, humectant, keratolytic, moisturizer, straightening agent, oxidizer, mineral and organic particle, plastic, polymer, opacifying agent, perfume, pH adjuster, preservative, protein, retinoid, reducing agent, sequesterant, skin bleaching agent, skin conditioning agent, skin smoothing agent, skin soothing agent, skin healing agent, softener, solubilizing agent, surfactant, lubricant, thickener, penetrant, permeation enhancer, analgesic, anti-inflammatory agent, antibiotic, anesthetic, plasticizer, salt, solvent essential oil, sunscreen and UV-absorber, vitamin, provitamine, plant extract, ceramide and pseudoceramide.

In another instance, the cosmetic composition comprises a foundation, mascara, eye liner, concealer, lip coating, blush for cheeks or eyelids, body makeup or nail coating.

In another instance, the cosmetic composition is formulated in the form of a liquid, solid, cream, ointment, solution, gel, mousse, stick, spray, powder, emulsion or dispersion.

In yet another instance, a film forming composition having improved pliability comprises at least one POSS having a three-dimensional cage structure formed of a plurality of Si subunits, at least one of said Si subunits having one or more R groups; at least one silicone resin other than a POSS, wherein, if said silicone resin comprises a polymethylsilsesquioxane, then said POSS comprises other than 8 Si subunits, or at least one of said R groups of said POSS is other than a methyl group, or said POSS has the structure of an incomplete cage, wherein said composition has improved pliability over film forming compositions containing a mixture of polymethylsilsesquioxane POSS and non-POSS silicone resins.

In another instance, the ratio of said POSS to said silicone resin comprises from 1:4 to about 1:1.

In another instance, the ratio of said POSS to said silicone resin comprises from about 1:3 to about 9:11.

In another instance, the ratio of said POSS to said silicone resin comprises from about 3:7 to about 2:3.

In another instance, the POSS is a liquid at room temperature.

In another instance, a cosmetic composition comprises a solvent and a colorant, wherein the total amount of the POSS and silicone resin comprises from about 0.5% to about 35% by weight of said cosmetic composition.

In another instance, the cosmetic composition comprises a foundation, mascara, eye liner, concealer, lip coating, blush for cheeks or eyelids, body makeup or nail coating.

In yet another instance, a cosmetic composition having improved wear comprises at least one POSS having a three-dimensional cage structure formed of a plurality of Si subunits, at least one of said Si subunits having one or more R groups; at least one silicone resin other than a POSS, wherein, if said silicone resin comprises a polymethylsilsesquioxane, then said POSS comprises other than 8 Si subunits, or at least one of said R groups of said POSS is other than a methyl group, or said POSS has the structure of an incomplete cage; a solvent; and a coloring agent, wherein said cosmetic composition has improved wear over cosmetic compositions containing a mixture of polymethylsilsesquioxane POSS and non-POSS silicone resins.

In another instance, the total amount of said POSS and non-POSS silicone resin comprises from about 0.5% to about 35% by weight of said cosmetic composition.

In another instance, the ratio of said POSS to said silicone resin comprises from about 1:4 to about 1:1.

In another instance, the ratio of said POSS to said silicone resin comprises from about 1:3 to about 9:11.

In another instance, the ratio of said POSS to said silicone resin comprises from about 3:7 to about 2:3.

In another instance, the cosmetic composition comprises a foundation, mascara, eye liner, concealer, lip coating, blush for cheeks or eyelids, body makeup or nail coating.

In yet another instance, a process of making a cosmetic composition, comprises predetermining an amount of at least one POSS and at least one non-POSS silicone resin to impart improved wear or flexibility to said composition, said POSS having a three dimensional cage structure formed of a plurality of Si subunits, at least one of said Si subunits having one or more R groups, wherein, if said non-POSS silicone resin comprises a polymethylsilsesquioxane, then said POSS comprises other than 8 Si subunits, or at least one of said R groups of said POSS is other than a methyl group, or said POSS has the structure of an incomplete cage; and combining said amount of POSS, non-POSS silicone resin and at least one cosmetic ingredient.

In another instance, the step of predetermining said amounts further comprising preparing a film forming composition from said at least one POSS and said at least one non-POSS silicone resin, applying said film forming composition to a substrate and forming a film thereon; and testing the brittleness of said film.

In another instance, the at least one cosmetic ingredient comprises a solvent.

In another instance, the R group of said POSS is a hydrogen, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻N⁺(CH₃)₃, —OH, —(CH₂)ₙN⁺H₃X⁻ wherein n is 0-30 and X is a counter ion,

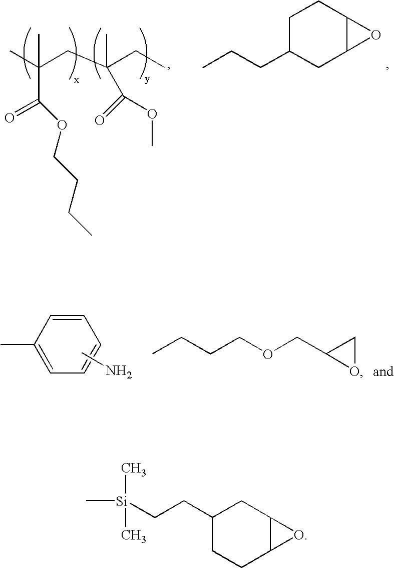

In another instance, the non-POSS silicone resin is an MQ resin.

In another instance, the non-POSS silicone resin comprises a plurality of Si subunits, at least one of said subunits of said silicone resin having an R group being an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin.

In another instance, the at least one silicone resin is trimethylsyloxysilicate.

In another instance, the at least one POSS is an Isooctyl-POSS.

In another instance, the cosmetic ingredient comprises a solvent, absorbent, alphahydroxy acid, betahydroxy acid, polyhydroxy acid, antiacne agent, anticaking agent, antifoaming agent, antimicrobial agent, antioxidant, astringent, binder, bleaching agent, buffer, biological additive, bulking agent, carrier, chelating agent, coupling agent, compatibilizer, conditioner, colorant, cosmetic astringent, cosmetic biocide, denaturant, drug astringent, detergent, deodorant, dispersant, external analgesic, emulsifier, film former, foaming agent, fragrance and fragrance component, humectant, keratolytic, moisturizer, straightening agent, oxidizer, mineral and organic particle, plastic, polymer, opacifying agent, perfume, pH adjuster, preservative, protein, retinoid, reducing agent, sequesterant, skin bleaching agent, skin conditioning agent, skin smoothing agent, skin soothing agent, skin healing agent, softener, solubilizing agent, surfactant, lubricant, thickener, penetrant, permeation enhancer, analgesic, anti-inflammatory agent, antibiotic, anesthetic, plasticizer, salt, solvent essential oil, sunscreen and UV-absorber, vitamin, provitamine, plant extract, ceramide and pseudoceramide.

In another instance, the cosmetic composition comprises a foundation, mascara, eye liner, concealer, lip coating, blush for cheeks or eyelids, body makeup or nail coating.

In another instance, the cosmetic composition is formulated in the form of a liquid, solid, cream, ointment, solution, gel, mousse, stick, spray, powder, emulsion or dispersion.

### DETAILED DESCRIPTION

The present invention relates to film forming compositions and cosmetic compositions having improved wear and/or pliability properties. In particular, it has been found that by combining certain POSS and non-POSS silicone resins in particular amounts, improved wear and/or pliability can be achieved over previous silicone resin-containing cosmetic compositions. Typically, this advantageous combination will be formulated in a cosmetic composition that contains at least one solvent, and, preferably, a colorant, such as a pigment. Other cosmetic ingredients known to those of skill in the art can also be combined therewith, depending on the desired product, such as a foundation, mascara, lip coating (lip stick, lip gloss, lip pencil and the like), nail coating and the like.

Silicone Resins

Silicone resins are named in accordance with what is referred to in the art as “MDTQ” nomenclature, whereby a silicone resin is described depending upon the various monomeric siloxane subunits (“Si subunits”) which form the polymer. Each letter, “M,” “D,” “T” and “Q” stands for a different subunit. “M” denotes the monofunctional unit (CH₃)₃SiO_{1/2}. This unit is referred to as monofunctional because the silicone atom shares only one oxygen with another Si atom in the chain. The “M” unit can be represented by the structure:

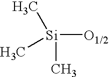

At least one of the methyl groups can be replaced, as demonstrated by the formula R(CH₃)₂SiO_{1/2}, where R can be a substituent other than a methyl group, as represented by the structure:

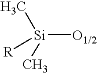

In fact, each methyl group can be replaced by an R group which may be the same or different. The letter “D” denotes the difunctional subunit (CH₃)₂SiO_{2/2} where two of the available bonds from the silicone atom are bound to oxygen in the formation of the polymeric chain. The “D” subunit can be represented as:

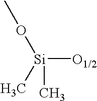

As was the case in connection with the M subunit, one or more methyl groups may be replaced with the same or different R groups. The symbol “T” denotes the trifunctional subunit, (CH₃)SiO_{3/2} and can be represented as:

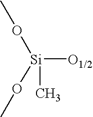

As illustrated above in connection with the “M” subunit, any methyl group can be replaced in the “T” subunits with another functional or R group.

Finally, the symbol “Q” denotes the quadrifunctional subunit SiO_{4/2} which can be represented as:

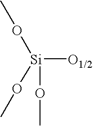

From this description, it is apparent that by varying the number and types of subunit, “M,” “D,” “T” and “Q,” and by varying the functional groups, a vast number of combinations can be created. As expected, the properties of each compound can also vary greatly depending upon the number and type of substituents, size of the polymeric chain, and the degree of bridging between the subunits.

In one embodiment, the non-POSS silicone resin of the invention is a Q resin. By the term Q resin, it is meant that the resin contains predominantly Si subunits of the Q type, or that those of skill in the art would regard the particular resin predominantly as a Q resin. MQ resins are also referred to as “siloxysilicates”, such as trimethylsiloxysilicates, represented by the following formula: [(CH₃)₃—Si—O]ₓ—(SiO_{4/2})_{y} (MQ Units) where x and y can have values ranging from 50 to 80. In a further embodiment, the non-POSS silicone resin is a siloxysilicate chosen from any combination of M and Q units, for example, [(R)₃—Si—O]ₓ—(SiO_{4/2})_{y}, wherein x and y can have values ranging from 50 to 80 and at least one R group is chosen from an alkyl group other than a methyl or longer hydrocarbon chain. For example, a Q resin can be chosen from among the Wacker 803 and 804 resins, available from Wacker Silicone Corporation, and G.E. 1,170-002, available from General Electric.

The term “silsesquioxane” refers to a class of silicone resins of the T type (“T resins”). In one embodiment, the silicone resin can be chosen from silsesquioxanes represented by the following formula: (CH₃SiO_{3/2})ₓ (T Units) where x has a value of up to several thousand and the methyl may be replaced by another R group as described above for the M subunits. Note, however, that where a polymethylsilsesquioxane is employed, it is not combined with a POSS of the type having only 8 fully saturated Si subunits (complete cage of R₁ to R₈ methyl groups). Polymethylsilsesquioxanes are those silsesquioxanes wherein each substituent (R group) is a methyl group.

In another embodiment of the present invention the non-POSS silicone resin is a “polyalkylsiloxane” or D resin. Again, by the term “D resin” it is meant that the resin contains predominantly Si subunits of the D type, or that those of skill in the art would regard the particular resin predominantly as a D resin. In one embodiment, the polysiloxane is of the formula [R₃—Si—O]—(R₂SiO)_{X}—[Si—R₃], wherein X is at least 2000, and R is an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin. D resins include dimethylsiloxanes having the CTFA designation dimethicone. These siloxanes are available commercially from the General Electric Company as the Viscasil Series and from Dow Corning as the DC200 series.

Substituting Silicone Resins

As stated previously, the M, D and T subunits may include one or more substituents (R-groups). As non-limiting examples, these R groups may include an alkyl, hydroxyl, alkoxysilane, amine, chlorosilane, epoxide, ester, halide, methacrylate, molecular silica, nitrile, norborene, olefin, phosphine, silane, silanol, styrenic polymer or polyolefin.

More than one substitution can be made, wherein two or more of the methyl groups available are replaced with the same or different R groups. These groups can be directly bonded to the Si atom, or may be bound through a bridging molecule such as an azo, diazo, epoxy or halogen bond.

POSS

The term “POSS” stands for Polyhedral Oligomeric SilSesquioxane(s). However, not all Polyhedral Oligomeric SilSesquioxanes qualify as “POSS” for purposes of the invention. In general, POSS refers to only those compounds existing in a rigid, “CAGE”-type configuration, examples of which are shown in Formulas I-V, below.

Among the POSS resins, only certain structures, such as, by way of non-limiting examples, those illustrated in Formulas I, III and IVA are referred to herein as being “complete cages” wherein all of the sides of the three-dimensional structure are completed sides and all of the Si atoms are completely saturated.

In particular, the POSS molecules included in the compositions of the present invention are distinguishable from other Polyhedral oligomeric SilSesquioxanes that can exist, for example, in the ladder configuration of Formula VI.

For example, the polymethylsilsesquioxane known as Resin MK (also known as SiliconHarz MK, and BELSIL® PMS MK), which may have been sold by Wacker Chemical Corp., 3306 Sutton Road, Adrian, Mich. 49221, has previously been disclosed in connection with cosmetic formulations in U.S. Patent Application Publication No. US2002/0114773A1 (published Aug. 22, 2002). As disclosed therein, the belief is that the compounds exist in both a “cage” (i.e., Formula I, wherein R₁-R₈ are CH₃—) and “ladder” configuration (Formula VI). It is also believed that the majority of the silicone polymers are present in the ladder configuration (Formula VI). To the extent this composition contains the “ladder” configuration, it is not POSS.

In order to form the three-dimensional cage structure, a POSS in accordance with the invention must have at least 6 Si molecules. In a preferred embodiment of the present invention, POSS contains 8 Si atoms. POSS may also include greater than 8 Si atoms. The number of Si atoms can range from 9 to 100, alternatively 9 to 30, also alternatively 9 to 20 and finally alternatively 9 to 16. At least four of the Si atoms are bound, through an oxygen atom, to at least 3 other Si atoms (referred to herein as being “completely saturated”). All of the Si atoms are bound to at least one other Si atom through an oxygen bridge. As shown in the exemplary and non-limiting structures of Formulas I through V, POSS forms a rigid three-dimensional cage structure having at least two completed sides. This rigid cage structure is distinguished from ladders and other structures which are not held in place in three directions. See Formula VI. Each of the Si atoms is bound to at least 1 R group with no more than 1 Si atom being bound to more than 2 R groups. The POSS molecule illustrated in Figure III has six saturated Si atoms and 5 complete sides (2 sides bounded by 3 Si atoms connected through oxygen bridges and 3 sides bounded by 4 Si atoms connected through oxygen bridges). Formula II B has 4 such saturated Si atoms and 2 completed sides, (both bounded by 4 Si atoms connected through oxygen bridges). Formula II C has 6 saturated Si atoms and 3 completed sides all bounded by 4 Si atoms connected through oxygen bridges.

As stated above, POSS has previously been disclosed in connection with cosmetic formulations in the commonly owned U.S. patent applications entitled “POSS CONTAINING COSMETICS AND PERSONAL CARE PRODUCTS,” filed Mar. 12, 2004; and “EPOSS CONTAINING COSMETICS AND PERSONAL CARE PRODUCTS,” filed Mar. 12, 2004. Therein, POSS is referred to as having 8 Si atoms or less, while EPOSS includes those cage structures having greater than 8 Si atoms. The present invention makes no such distinction; all silicone resins forming the cage structure are considered “POSS,” regardless of the number of Si atoms.

POSS molecules in accordance with one of embodiment of the present invention have the complete cage structure of Formula I:

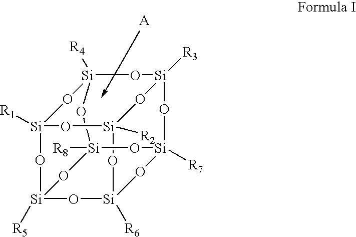

It is also possible that one or even two of the oxygen bridges between successive silicon atoms are broken or missing, in which case the “POSS” is referred to as having an “incomplete” case structure. By way of non-limiting examples, consider the rigid three-dimensional cage structures illustrated in Formulas IIA-E:

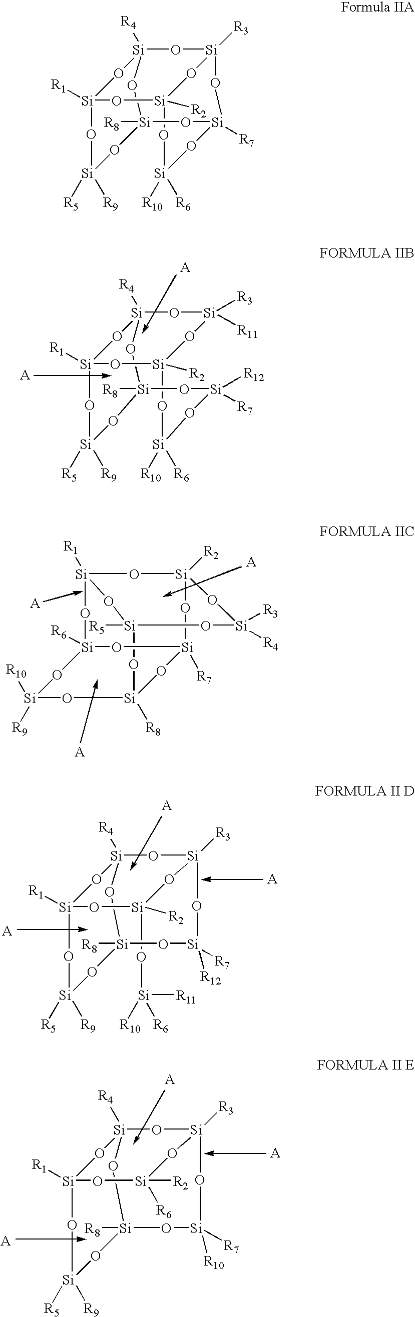

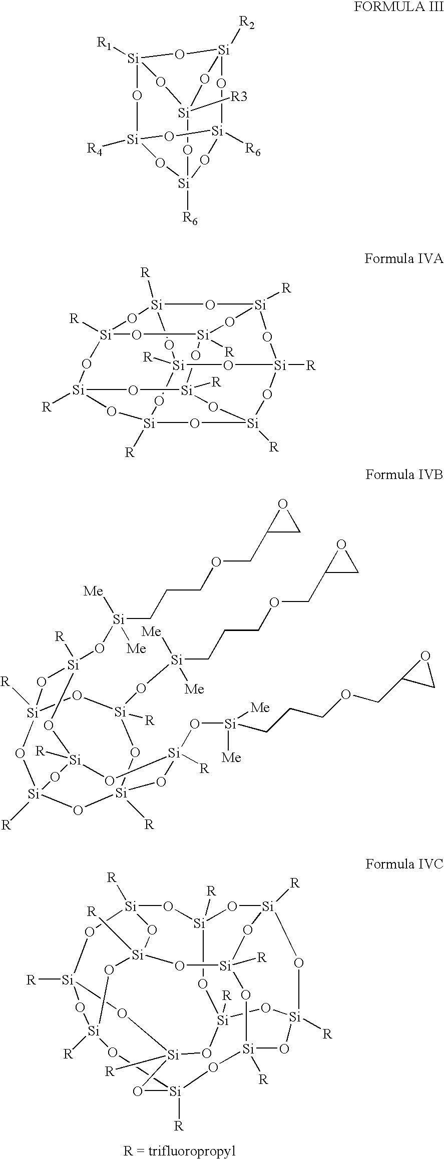

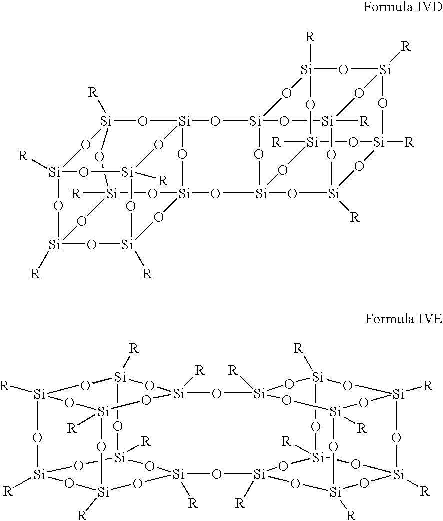

In Formula IVA, the number of Si atoms in the cage is 10, in Formula IVB, the number of Si atoms is 10 and in Formula IVC, the number of Si atoms in the cage is 12. In Formulae IVD and IVE, the number of Si atoms in the cage or core is 16. An additional example of an “incomplete” cage structure, wherein one or more of the oxygen bridges between successive silicon atoms is broken or missing, is illustrated in Formula V:

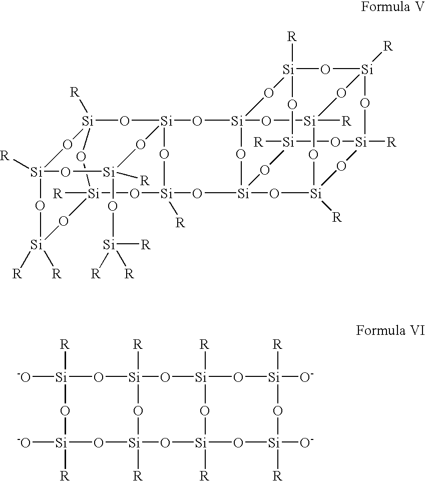

Formula III is a complete cage, but produced from six Si atoms. Formula VI, a ladder configuration (not a POSS according to the invention), can be a monomer linked end to end to other similar structures. It is not rigid within the meaning of this document as it can fold or flex around each R—Si—O—Si—R axis of the molecule. No such movement is possible in the rigid 3-D cage structures (whether complete or incomplete) of the invention. Thus, the molecules of this formula are not POSS.

Note also that when referencing POSS molecules as being in accordance with Formula II, having the structure of Formula II, or being other than a completed cage, the sides which are illustrated as “open,” “missing” or “broken” are illustrative only. When reference is made to Formula II, it is understood that any one or two sides, or any one or two oxygen bridges, may be broken or missing. The structure of the POSS molecule can be roughly thought of as a box (prism in the case of Formula III) or cage in shape with silicon (Si) atoms at each corner. Each Si atom is connected to at least one other Si atom through bonds to an oxygen atom (also referred to as an “oxygen bridge”). Preferably, at least four of the Si atoms in the POSS structure are “completely saturated.” Most preferably, all of the Si atoms are bound, through oxygen atoms, to three other Si atoms within the cage as shown in Formulas I, III and IVA, thus all the Si atoms are “completely saturated.” While illustrated in Formula I as Si atoms, the groups at each corner may be the same or different and may be one or more atoms or groups including, without limitation, silicon, silane, siloxane, silicone or organometallic groups. The POSS of the invention also exists in a rigid 3-dimensional cage structure as illustrated, for example, in Formulas I-V and the cage has at least two completed sides A. Each Si is bound to at least one R group with no more than one Si atom being bound to more than two R groups.

In one embodiment, the POSS will be a liquid at room temperature.

Substituting POSS

Similar to other silicone resins, the M, D or T subunits of a POSS can be “derivatized” by the replacement of a methyl group with a functional group other than a methyl. As non-limiting examples, one or more methyl groups could be replaced with another alkyl group, alkene, alkyne, hydroxyl, thiol, ester, acid, ether. In one embodiment, the “IR groups” of the present invention include, without limitation, one or more of the following: methyl, ethyl, propyl, isobutyl, isooctyl, phenyl, cyclohexyl, cyclopentyl, —Osi(CH3)2-CH2-CH2-(CF2)5CF3, —(CH2)3SH, N+(CH3)3, O—N+CH3)3, —OH, —(CH2)nN+H3X— wherein n is 0-30 and X is a counter ion,

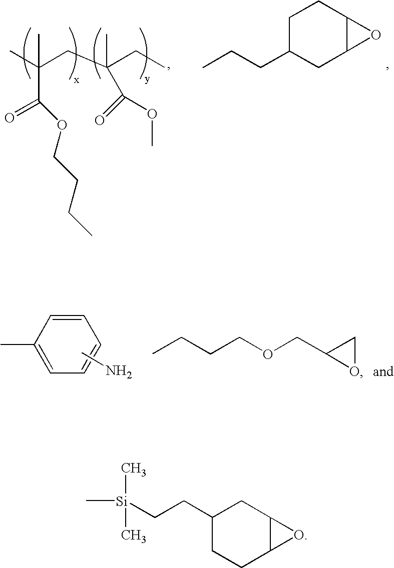

Preferably, the R group is an isooctyl group. These substituent groups may be bound directly to the cage structure or may be bound through a bridging molecule such as an azo, diazo, epoxy or halogen containing material.

For example, the one remaining bond of each silicon of Formula I, III and IVA can bind to a variety of substituents or groups specified, as “R” groups (R₁-R₈), ((R₁-R₆) in Formula III). In some embodiments illustrated in Formulas II, IVB and V a POSS molecule in which one or two of the oxygen bridges between adjacent silicon molecules have been eliminated, a greater number of R groups are possible. When a POSS having 8 Si atoms is employed, it is preferred that no more than two of these inter-silicon connections (oxygen bridges) be eliminated. However, it is possible to eliminate as many as three such bridges (Formula IIE). More preferably, only a single oxygen bridge would be eliminated (Formula IIA). As stated above, the Si molecules not completely bound may have one or more additional positions available for binding additional substituents. In the case of a single missing side, the POSS molecule may include additional R group R₉ and R₁₀, which may be the same or different as the R group R₁-R₈. When 2 or 3 bridges are missing, the POSS molecule may include additional R groups R₉, R₁₀, R₁₁ and R₁₂ (as appropriate), which all may be the same or different and may be the same as the groups identified for R1-R₈.

POSS compounds with various R groups are well known in the literature. They are described in a number of patents including, without limitation, Weidner et al., U.S. Pat. No. 5,047,492, issued Sep. 10, 1991; Sojka, U.S. Pat. No. 5,389,726, issued Feb. 14, 1995; Lichtenhan et al., U.S. Pat. No. 5,484,867, issued Jan. 16, 1996; Lichtenhan et al., U.S. Pat. No. 5,589,562, issued Dec. 31, 1996; Crocker et al., U.S. Pat. No. 5,750,741, issued May 12, 1998; Banaszak Holl et al., U.S. Pat. No. 5,858,544, issued Jan. 12, 1999; Lichtenhan et al., U.S. Pat. No. 5,939,576, issued Aug. 17, 1999; Lichtenhan et al., U.S. Pat. No. 5,942,638, issued Aug. 24, 1999; Lichtenhan et al., U.S. Pat. No. 6,100,417, issued Aug. 8, 2000; Van Santen et al., U.S. Pat. No. 6,127,557, issued Oct. 3, 2000; Takamuki et al., U.S. Pat. No. 6,207,364, issued Mar. 27, 2001; Zank et al., U.S. Pat. No. 6,252,030, issued Jun. 26, 2001; Nguyen, U.S. Pat. No. 6,270,561, issued Aug. 7, 2001; Mehl et al., U.S. Pat. No. 6,277,451, issued Aug. 21, 2001; Lichtenhan et al., U.S. Pat. No. 6,362,279, issued Mar. 26, 2002; and Barbee et al., U.S. Pat. No. 6,486,254, issued Nov. 26, 2002. These patents describe in detail various methods of producing the basic POSS cage structure and various derivatives thereof, including POSS based polymers. To the extent that these patents identify and describe various POSS molecules having the structures of Formula I-V, derivatives and polymers thereof, they are incorporated by reference. The discussions of techniques for manufacturing and derivatizing this class of compounds described in each of these patents is also hereby incorporated by reference.

In general, R groups (for example, R₁, R₂, R₃, R₄, R₅, R₆, R₇, R₈, R₉, R₁₀, R₁₁ and R₁₂ as shown in the figures and any other R groups appropriate) can be the same or different and may be reactive or nonreactive groups. They may be, in replacing a methyl or H, for example, hydroxy (—OH), alkane derivatives (missing a hydrogen) also known as alkyl groups (other than methyl), alkenyl groups also referred to as derivatives of alkenes (having one or more double bonds), usually missing an H where they are bound to Si in POSS or to some other molecule, alkynyl groups also referred to as derivatives of alkynes (having one or more triple bonds) usually missing an H where they are bound to Si in POSS or to some other molecule, aryl groups (either the 6-carbon ring of benzene or the condensed 6-carbon rings of other aromatic derivatives such as naphthalene) also referred to as derivatives of arenes, usually missing an H where they are bound to Si in POSS or to some other molecule, acyl groups (organic acids without the OH group, e.g., CH₃CO— or C₆H₅CO—), alkoxy groups (alkyl radicals attached to the remainder of a molecule by oxygen), such as methoxy, ester groups, acid groups, acrylate groups, alkyl acrylate groups, hydroxy groups, halogens, amino groups, alkylamino groups, aminoalkyl groups, groups containing one or more tertiary or quaternary nitrogens, silicone containing groups, sulfur containing groups, epoxides, azo groups, diazo groups, halogens, cyclic compounds which can undergo ring opening polymerization or ring opening metathesis polymerization. R groups may also be monomers or polymers where POSS will be used as a pendant substituent of the polymer. Acrylates and cationic polymers providing conditioning properties are provided in one embodiment.

Where appropriate, any of these R groups may themselves be substituted or unsubstituted, saturated or unsaturated, linear or branched. Possible substitutions include C₁-C₃₀ alkyl groups, C₁-C₃₀ alkenyl groups, C₁-C₃₀ alkynyl groups, C₆-C₁₈ aryl groups, acyl groups, alkoxy groups, carboxy groups, ester groups, acrylate groups, alkyl acrylate groups, trihydroxy groups, amino groups, alkylamino groups including mono and dialkylamino groups, mono and dihydroxy alkylamino groups, cyano groups, aminoalkyl groups, groups containing one or more tertiary or quaternary nitrogens, silicone containing groups, sulfur and/or phosphorous containing groups, SO₂X, SO₃X, where X is H, methyl or ethyl, epoxides, azo groups, diazo groups, halogens, cyclic compounds which can undergo ring opening polymerization or ring opening metathesis polymerization. Indeed, any group which can be attached to a corner of a POSS molecule can be used.

When these R groups are carbon containing fatty acids or fatty alcohols, aromatic or cyclic groups, they generally may contain between six and 50 carbon atoms and may be saturated or unsaturated, substituted as discussed above or unsubstituted and branched or linear, as appropriate for a given group.

More specifically, possible R groups include, without limitation, hydroxy groups including mono or poly hydroxy groups, phenols, alkoxy, hydroxy alkyls, silanes, amino and in particular, quats, halosilanes, epoxides, alkyl carbonyls, alkanes, haloalkyls, halogens, acrylates, methacrylates, thiols, nitriles, norbornenyls, branched alkyl groups, polymers, silanes, silanols, styryls and thiols. In a single POSS molecule of Formula I, R₁ could be H, R₂ —OH, R₃ —NH₂, R₄ —CH₂CH₂N+CH₃(OCH₂CH₃)CH₂CH₂CH₃, R₅ —CH₂CH₂CHOCH₂ (epoxide), R₆ —OC(CH₃)₃, R₇ —OOC(CH₂)₁₆CH₃ and R₈ could be Cl. This is a hypothetical example, merely to illustrate that each of the R groups can be derivatized separately and to emphasize the wide variety of possible substitutions.

In one embodiment, these POSS molecules are not completely substituted with the same R groups (e.g., not all R₁-R₆, R₁-R₈, R₁-R₁₀ or R₁-R₁₂ (and any other R groups, as appropriate, given the number of Si atoms and available bonds in a given POSS molecule) are methyl, isobutyl or phenyl). This is particularly preferred for POSS molecules that have the structure of Formula I. Moreover, when a POSS molecule having 8 Si subunits, as depicted in Formula I, is employed, at least one of the R groups is a group other than a methyl, particularly where the non-POSS silicone resin is a T resin and, even more particularly, Resin MK.

Also contemplated under the term POSS is the family of commercially available compounds available from Hybrid Plastics, 18237 Mount Baldy Circle, Fountain Valley, Calif. 92708-6117 and Mayaterials, Inc. P.O. Box 87, South Lyon, Mich. 48178-0087. According to the manufacturers, these commercially available materials break down into several general categories:

POSS Molecular Silicas™

POSS Molecular Silicas™ possess a robust Si—O core surrounded by non-reactive organic groups (R1-R8) which permit the inorganic core to be compatible with an organic matrix. This allows POSS Molecular Silicas™ to be compounded into standard polymers yielding true nanocomposites with complete molecular level dispersion. The unique ability of POSS Molecular Silicas™ to be dispersed at the molecular level is the key to reinforcing polymer segments and coils leading to significant property enhancements.

POSS Silanols

POSS Silanols possess a hybrid inorganic-organic three-dimensional structure which contains from one to four stable silanol (Si—OH) groups.

POSS Functionalized Monomers

POSS Functionalized Monomers possess a hybrid inorganic-organic three-dimensional structure which contains from one to eight reactive organic functional groups. The majority of POSS Functionalized Monomers contain seven non-reactive organic groups with one unique functionality. The unique functional groups that are currently available include, but are not limited to, amines, esters, epoxides, methacrylates, olefins, silanes, styryls, and thiols. By varying the functional group and the seven non-reactive organic groups, a multitude of POSS Functionalized Monomers can be prepared to meet almost any need. While the monofunctional POSS Monomers can be incorporated by copolymerization or grafting, multifunctional POSS Monomers can be utilized as effective cross-linkers. POSS Functionalized Monomers react similarly in polymerization, grafting and cross-linking reactions to standard organic monomers. While they react like simple organic monomers, when incorporated into a polymeric material, POSS Functionalized Monomers impart significant improvements in the thermal, mechanical, and gas separation properties of traditional plastics.

POSS Polymers and Resins

POSS Polymers and Resins possess a hybrid inorganic-organic composition and can be either thermoplastic or thermoset materials. As a class of materials, POSS Polymers and Resins are comprised of either (1) polymers in which a POSS Functionalized Monomer has been co-polymerized or grafted onto a polymer chain, or (2) silsesquioxane resins possessing some cage structure. POSS Polymers and Resins can be used as stand alone replacements for traditional materials or they may be compounded or solution blended into traditional polymeric materials to enhance the properties of the base resin. The types of POSS Polymers and Resins that are currently available include, but are not limited to, silicones, styrenics, acrylics, and norbornenes.

POSS molecules available from Hybrid Plastics include, without limitation, those based on Formulas I-IV, and are selected from alcohols and phenols such as (TMP DiolCyclopentyl-POSS, TMP DiolIsobutyl-POSS, Trans-CyclohexaneDiolCycohexyl-POSS, Trans-CyclohexaneDiolIsobutyl-POSS, 1,2-PropaneDiolCyclohexyl-POSS, 1,2-PropaneDiolIsobutyl-POSS, and OctaHydroxypropyldimethylsilyl-POSS), alkoxysilanes such as (DiethoxymethylsilylethylCycohexyl-POSS, DiethoxymethylsilylethylIsobutyl-POSS, DiethoxymethylsilylpropylCyclohexyl-POSS, DiethoxymethylsilylpropylIsobutyl-POSS, EthoxydimethylsilylethylCyclohexyl-POSS, EthoxydimethylsilylethylIsobutyl-POSS, EthoxydimethylsilylpropylCyclohexyl-POSS (may contain some isomer), EthoxydimethylsilylpropylIsobutyl-POSS (may contain some β-isomer), TriethoxysilylethylCyclohexyl-POSS, TriethoxysilylethylIsobutyl-POSS, TriethoxysilylpropylCyclohexyl-POSS (may contain some β-isomer) and TriethyoxysilylpropylIsobutyl-POSS (may contain some β-isomer), amines such as (AminopropylCyclohexyl-POSS, AminopropylIsobutyl-POSS, AminopropylIsooctyl-POSS, AminoethylaminopropylCyclohexyl-POSS, AminoethylaminopropylIsobutyl-POSS, OctaAminophenyl-POSS and OctaAmmonium-POSS), chlorosilanes such as (MonoChloroCyclohexyl-POSS, MonoChloroCyclopentyl-POSS, MonoChloroIsobutyl-POSS, ChlorodimethylsilylethylCyclohexyl-POSS, ChlorodimethylsilylethylIsobutyl-POSS, ChlorodimethylsilylpropylCyclohexyl-POSS (may contain some β-isomer), chlorodimethylsilylpropylIsobutyl-POSS (may contain some β-isomer), DichloromethylsilylethylCyclohexyl-POSS, DichloromethylsilylethylIsobutyl-POSS, DichloromethylsilylpropylCyclohexyl-POSS (may contain some β-isomer), DichloromethylsilylpropylIsobutyl-POSS (may contain some β-isomer), TrichlorosilylethylCyclohexyl-POSS, TrichlorosilylethylIsobutyl-POSS, TrichlorosilylpropylCyclohexyl-POSS (may contain some β-isomer), TrichlorosilylpropylIsobutyl-POSS (may contain some β-isomer), Octa(chlorosilylethyl)-POSS (may contain some β-isomer), Octa(dichlorosilylethyl)-POSS (may contain some β-isomer) and Octa(trichlorosilylethyl)-POSS (may contain some β-isomer), epoxides such as (EpoxyCyclohexylCyclohexyl-POSS, EpoxyCyclohexylCyclopentyl-POSS, EpoxyCyclohexylIsobutyl-POSS, EpoxyCyclohexylDisilanolIsobutyl-POSS, EpoxyCyclohexyl-POSS Cage Mixtures such as EpoxypropylCyclopentyl-POSS, EpoxypropylIsobutyl-POSS, GlycidylCyclohexyl-POSS, GlycidylCyclopentyl-POSS, GlycidylEthyl-POSS, GlycidylIsobutyl-POSS, GlycidylIsoctyl-POSS, GlycidylPhenyl-POSS, OctaEpoxyCyclohexyldimethylsilyl-POSS, OctaGlycidyldimethylsilyl-POSS, TrisGlycidylCyclohexyl-POSS, TrisGlycidylCyclopentyl-POSS, TrisGlycidylEthyl-POSS and TrisGlycidylIsobutyl-POSS), esters such as (EthylUndecanoateCyclohexyl-POSS, EthylUndecanoateCyclopentyl-POSS, EthylUndecanoateIsobutyl-POSS, MethylPropionateCyclohexyl-POSS, MethylPropionateCyclopentyl-POSS and MethylPropionateIsobutyl-POSS), fluoroalkyls such as (Fluoro(3)DisilanolCyclopentyl-POSS, Fluoro(13)DisilanolCyclopentyl-POSS, Fluoro(13)DisilanolIsobutyl-POSS, MethacrylFluoro(3)Cyclopentyl-POSS mixture of isomers, MethacrylFluoro(13)Cyclopentyl-POSS mixture of isomers, MethacrylFluoro(3)Isobutyl-POSS mixture of isomers, DodecaTrifluoropropyl-POSS, TriFluoroCyclohexyl-POSS, TriFluoroCyclopentyl-POSS, TriFluoroIsobutyl-POSS, TrifluoropropylIsobutyl-POSS, TrisFluoro(3)Cyclopentyl-POSS and TrisFluoro(13)Cyvlopentyl-POSS), halides such as (ChlorobenzylCyclohexyl-POSS, ChlorobenzylCyclopentyl-POSS, ChlorobenzylIsobutyl-POSS, ChlorobenzylethylCyclohexyl-POSS, ChlorobenzylethylCyclopentyl-POSS, ChlorobenzylethylIsobutyl-POSS, ChlorophenylCyclohexyl-POSS, ChlorophenylCyclopentyl-POSS, ChlorophenylIsobutyl-POSS, ChlorophenylPhenyl-POSS, ChloropropylCyclohexyl-POSS, ChloropropylCyclopentyl-POSS and ChloropropylIsobutyl-POSS), isocyanates such as (IsocyanatopropyldimethylsilylCyclohexyl-POSS and IsocyanatopropyldimethylsilylIsobutyl-POSS), methacrylates & acrylates such as (AcryloCyclohexyl-POSS, AcryloCyclopentyl-POSS, AcryloIsobutyl-POSS, MethacrylCyclohexyl-POSS, MethacrylCyclopentyl-POSS, MethacrylEthyl-POSS, MethacrylIsobutyl-POSS, MethacrylIsooctyl-POSS, 90%, MethacrylPhenyl-POSS, MethacrylDisilanolCyclohexyl-POSS, MethacrylDisilanolCyclopentyl-POSS, MethacrylDisilanolIsobutyl-POSS, MethacrylFluoro(3)Cyclopentyl-POSS, MethacrylFluoro(13)Cyclopentyl-POSS, MethacryltrimethylsiloxyCyclopentyl-POSS, MethacryltrimethylsiloxyIsobutyl-POSS, Methacryl-POSS Cage Mixture, OctaMethacryldimethylsilyl-POSS, TrisMethacrylCyclohexyl-POSS and TrisMethacrylIsobutyl-POSS), molecular silica such as (DodecaPhenyl-POSS, DodecaPhenyl-POSS, 85%, Isooctyl-POSS Cage Mixture, 95%, OctaCyclohexyl-POSS, OctaCyclopentyl-POSS, OctaIsobutyl-POSS, OctaMethyl-POSS, OctaPhenyl-POSS, OctaTMA-POSS, DodecaTrifluoropropyl-POSS, OctaTrimethylsiloxy-POSS, Phenethyl-POSS Cage Mixture and PhenethylIsobutyl-POSS), nitriles such as (CyanoethylCyclohexyl-POSS, CyanoethylCyclopentyl-POSS, CyanoethylIsobutyl-POSS, CyanopropylCyclohexyl-POSS, CyanopropylCyclopentyl-POSS and CyanopropylIsobutyl-POSS), norbornenyls such as (NorbornenylethylCyclohexyl-POSS, NorbornenylethylIsobutyl-POSS, NorbornenylethylDiSilanolCyclohexyl-POSS, NorbornenylethylDiSilanolCyclopentyl-POSS, NorbornenylethylDiSilanolIsobutyl-POSS, Tris NorbornenylCyclohexyl-POSS, Tris NorbornenylCyclopentyl-POSS and Tris NorbornenylIsobutyl-POSS), olefins such as (AllylCyclohexyl-POSS, AllylCyclopentyl-POSS, AllyIsobutyl-POSS, AllylDimethylsilylCyclopentyl-POSS, CyclohexenylethylCyclopentyl-POSS, DimethylvinylCyclopentyl-POSS, DiphenylvinylCyclopentyl-POSS, MonoVinylCyclohexyl-POSS, MonoVinylCyclopentyl-POSS, MonoVinylIsobutyl-POSS, PhenylMethylVinylCyclopentyl-POSS, Tris(Dimethylvinyl)Isobutyl-POSS, TrivinylsilylCyclopentyl-POSS, OctaCyclohexenyldimethylsilyl-POSS, OctaVinyldimethylsilyl-POSS, OctaVinyl-POSS and Vinyl-POSS Cage Mixture), phosphines such as (DiphenylphosphinoethylCyclopentyl-POSS and DiphenylphosphinopropylCyclopentyl-POSS), polymers such as (Poly(dimethyl-co-methylhydrido-co-methylpropylPOSS)siloxane, Poly(dimethyl-co-methylvinyl-co-methylethylsiloxyPOSS)siloxane, OctaMethyl-POSS Nanoreinforced™ Polypropylene, 10 wt %, Poly(ethylsilsesquixane) uncured, Poly(methylsilsesquioxane) uncured, Poly(phenylsilsesquioxane) uncured, Poly(propylmethacrylPOSS-co-methylmethacrylate), Poly(propylmethacrylPOSS-co-styrene), Poly(styrylPOSS-co-styrene), Poly(vinylsilsesquioxane) uncured and Poly(vinylsilsesquioxane) fully cured FireQuench™), silanes such as (DimethylsilaneCyclohexyl-POSS, DimethylsilaneCyclopentyl-POSS Schwab Hydride, DimethylsilaneIsobutyl-POSS, MonoSilaneCyclohexyl-POSS, MonoSilaneIsobutyl-POSS, OctaSilane-POSS, Tris(Dimethylsilane)Cyclohexyl-POSS, Tris(Dimethylsilane)Cyclopentyl-POSS and Tris(Dimethylsilane)CycloIsobutyl-POSS), silanols such as (CyclohexenyldimethylsilyDisilanolIsobutyl-POSS, DimethylphenylDisilanolCyclopentyl-POSS, DimethylvinylDisilanolCyclohexyl-POSS, DimethylvinylDisilanolCyclopentyl-POSS, DimethylvinylDisilanolIsobutyl-POSS, DiSilanolCyclopentyl-POSS, DiSilanolIsobutyl-POSS, EpoxyCyclohexylDisilanolIsobutyl-POSS, Fluoro(3)DisilanolCyclopentyl-POSS, Fluoro(13)DisilanolCyclopentyl-POSS, Fluoro(13)DisilanolIsobutyl-POSS, MethacrylDisilanolCyclohexyl-POSS, MethacrylDisilanolCyclopentyl-POSS, MethacrylDisilanolIsobutyl-POSS, MonoSilanolCyclohexyl-POSS, MonoSilanolCyclopentyl-POSS Schwabinol, MonoSilanolIsobutyl, NorbornenylethylDiSilanolCyclohexyl-POSS, NorbornenylethylDiSilanolCyclopentyl-POSS, NorbornenylethylDiSilanolIsobutyl-POSS, TMS DiSilanolCyclohexyl-POSS, TMS DiSilanolIsobutyl-POSS, TriSilanolCyclohexyl-POSS, TriSilanolCyclopentyl-POSS, TirSilanolEthyl-POSS, TriSilanolIsobutyl-POSS, TriSilanolIsooctyl-POSS and TriSilanolPhenyl-POSS), styrenes such as (StyrenylIsobutyl-POSS, StyrylCyclohexyl-POSS, StyrylCyclopentyl-POSS and StyrylIsobutyl-POSS), and thiols such as (MercaptopropylCyclohexyl-POSS, MercaptopropyIsobutyl-POSS and MercaptopropylIsooctyl-POSS, 90%). Other POSS products may be purchased from ALDRICH. Still others are described in Weidner et al., U.S. Pat. No. 5,047,492 issued Sep. 10, 1991, the text of which, and in particular, the POSS molecules described in the passage of column 1, line 22 through column 2, line 48, are hereby incorporated by reference and Barry et al. U.S. Pat. No. 2,465,188, issued Mar. 27, 1948, the text of which is also hereby incorporated by reference. See also Banaszak Holl et al., U.S. Pat. No. 5,858,544, issued Jan. 12, 1989, the text of which is also incorporated by reference.

Particularly preferred POSS molecules useful for producing cosmetic compositions in accordance with the present invention include: TrisFluoro(13)Cyclopentyl-POSS (Cat. No. FL0590; C65H93F39O12Si10; MW: 2088.24 g/mole); MercaptopropylIsobutyl-POSS (Cat. No. TH1550; C31H70O12Si8; MW: 891.63 g/mole); MercaptopropylIsooctyl-POSS (Cat. No. TH1555; C59H126O12Si8; MW: 1284.37 g/mole); Poly(methacrylpropylisooctylPOSS-co-methymethacrylate) 60% wt (Cat. No. PM1275.4-60; (R7O14Si8)60-co-(C5H8O2)40); Poly(MethacrylpropylisooctylPOSS-co-methylmethacrylate) 80% wt (Cat. No. PM1275.4-80; (R7O14Si8)80-co-(C5H8O2)20); OctaIsobutyl-POSS (Cat. No. MS0825; C32H72O12Si8; MW: 873.60 g/mole); OctaPhenyl-POSS (Cat. No. MS0840; C48H40O12Si8; MW: 1033.53 g/mole); Isooctyl-POSS Cage Mixture, 95% (Cat. No. MS0805; [Me3CCH2CH(Me)CH2]nTn n=8; C64H136O12Si8; MW: 1322.46 g/mole based on n=8); EpoxyCyclohexylCyclohexyl-POSS (Cat. No. EP0399; C50H90O13Si8; MW: 1123.93 g/mole); EpoxyCyclohexylIsobutyl-POSS (Cat. No. EP0402; C36H76O13Si8; MW: 941.66 g/mole); GlycidylCyclohexyl-POSS (Cat. No. EP0415; C48H88O14Si8; MW: 1113.89 g/mole); GlycidylIsobutyl-POSS (Cat. No. EP0418); C34H74O14Si8; MW: 931.63 g/mole); TrisGlycidylCyclohexyl-POSS (Cat. No. EP0421; C66H128O18Si10; MW: 1490.57 g/mole); and OctaEpoxyCyclohexyldimethylsilyl-POSS (Cat. No. EP0430; C80H152O28Si16; MW: 2011.41 g/mole); OctaAminophenyl-POSS (Cat. No. AM0280; C48H48N8O12Si8; MW: 1153.63 g/mole); OctaAminophenyl-POSS (Cat. No. AM0285; C24H72CL8N8O12Si8; MW: 1173.18 g/mole); and OctaTMA-POSS (Cat. No. MS0860; C32H96O20Si8.˜60H2O; MW: 2218.75 g/mole). These POSS molecules can be purchased from Hybrid Plastics, 18237 Mount Baldy Circle, Fountain Valley, Calif. 92708-6117, USA and Maya Materials.

As previously noted and as reflected in the patents and publications previously incorporated by reference, there are many known POSS molecules and many known ways to produce POSS compounds and various derivatives and polymers therefrom. In general, however, one process of producing POSS includes the following steps: a) providing a trifunctional polyhedral oligomeric silsesquioxane of the formula Si₇R7O₉(OA)₃, where OA is —OH, —OSb(CH₃)₄, —OSₙ(CH₃)₃, or —OT1, and R is an alkyl, alkenyl, aryl, alkoxy group or other R group described herein; and b) corner capping said trifunctional polyhedral silsesquioxane by reacting said trifunctional polyhedral silsequioxane with a compound of the formula M-Z to form a polyhedral oligomeric silsesquioxane having the formula Si₇R₇O₁₂M_{(z)}. M is a silane, siloxane or organometallic group and Z is a reactive group selected from the group consisting of chloride, bromide or iodide. The process further includes the step of adding silver perchlorate to a solution of the polyhedral oligomeric silsesquioxane in aqueous acetone to convert reactive group Z to an alcohol. See U.S. Pat. No. 5,484,867. POSS molecules may also be made as described in a paper entitled “Polyhedral Oligosilsesquioxanes and Heterosilsesquioxanes” by Frank J. Feher of the Department of Chemistry of the University of California at Irvine, Calif. 92697-2025, USA, available from Gelest, Inc., the test of which is hereby incorporated by reference.

Film Forming and Cosmetic Compositions

In one aspect, the present invention relates to a composition comprising at least one POSS and at least one non-POSS silicone resin in amounts that have been found to provide improved wear and/or pliability characteristics over previous compositions comprising silicone resins without such a combination. In one embodiment of this aspect of the present invention, the non-POSS silicone resin is a Q or MQ resin. In another embodiment of this aspect, the non-POSS silicone resin is a Q or MQ resin and the POSS is a derivatized POSS, having at least one substituent other than a methyl group, more particularly, the POSS being Iso-octyl POSS.

In one embodiment, the formulation may simply be a film-forming composition containing a combination of at least one POSS and at least one non-POSS silicone resin. However, in most instances, the composition will be a cosmetic composition, such as a lip coating (for example, a lipstick or lip balm), liners, mascara, concealer, blush for cheeks or eyelids, foundation, rouge or nail coating (for example, a nail polish, nail enamel, nail varnish, nail conditioners, or nail dye) which contains one or more cosmetic ingredients.

“Wear,” as used herein, refers to the length of time the consistency, coverage, texture, and/or color of a material remains unnoticeably different when compared to the time of application, as viewed by the naked eye of a normal observer. Wear may be evaluated by a test involving the application of a composition to human keratinous material, for example, skin (including lips) and evaluating the consistency, texture and color of the composition after an extended period of time. For example, the consistency, texture and/or color of a lip composition may be evaluated immediately following application and these characteristics may then be re-evaluated and compared after an individual has worn the lip composition for a certain amount of time. These characteristics may be evaluated with respect to other compositions, such as commercially available compositions, a control or standard. Indeed, improved may be measured against an identical formulation without the combination of a POSS and non-POSS resin.

For film forming compositions, the compositions of the present invention also show surprisingly improved pliability. “Pliability” is referred to herein synonymously with “flexibility,” and refers to the ability of a film to be stressed without breaking or flaking. Pliability is often evaluated by applying a composition to a substrate upon which a film is formed. The film is then subjected to various stresses and observed in comparison to a standard or other film-forming composition.

Often, pliability is determined for a particular product by testing only the film-forming elements of the composition, as the final product may not be amenable to such testing. Accordingly, for a cosmetic composition, improved flexibility may be measured by testing the film forming elements alone, while the composition will still be considered to have “improved pliability,” which can impact other aspects of the composition, such as wear, appearance, texture and feel.

Accordingly, it will be appreciated that once the appropriate amounts of POSS and non-POSS silicone resin have been determined, the combination can be added to a cosmetic composition along with additional cosmetic ingredients (discussed below) to form a cosmetic composition having “improved pliability.”

The term “improved” refers to the fact that the composition has either greater wear and/or better pliability than has been previously found in connection with other compositions containing a POSS, a non-POSS silicone resin, or a combination thereof.

To achieve the desired results, the combined amount of POSS and non-POSS silicone resin should be from about 0.5% to about 35% by weight of the total cosmetic composition. Further, the amount of non-POSS silicone resin should always be equal to or greater than the amount of POSS present in the formulation. For example, the ratio of said POSS to said silicone resin can be from about 1:4 to about 1:1. More particularly, the ratio is from about 1:3 to about 9:11, and even more particularly from about 3:7 to about 2:3. However, the amount of POSS should not be below about 20% of the POSS/non-POSS combination, as this results in a mixture that is too tacky, and unsuitable for most compositions.

It will be recognized by those of skill in the art that the percentages and ratios referred to above may vary depending on the silicone resins selected. As indicated previously, the properties of these compounds can vary greatly depending upon the number and types of subunits, and functional groups selected. Moreover, the percentages and ratios may vary depending upon the cosmetic product desired and the additional cosmetic ingredients contained therein.

In some instances, a plurality of POSS compounds and/or a plurality of non-POSS silicone resins can be included in the total composition. In such instance, the combined amounts of POSS can be employed in approximately the same percentages and in the same ratios as when a single POSS is present. Similarly, the combined amounts of a silicone resin can be present in the approximately same percentages and in the same ratios as when a non-POSS single silicone resin in present. Of course, this is provided that the desired degree of wear and/or pliability is obtained.

Cosmetic Products/Cosmetic Ingredients

The cosmetic compositions of the invention will often include one or more “cosmetic ingredients.” This may include a single additional ingredient such as a solvent, surfactant, colorant or the like. However, most frequently, cosmetic products are a mixture of cosmetic ingredients traditionally found in those products. For example, in the case of a mascara, these cosmetic ingredients could include one or more of the components of Maybelline Great Lash® Very Black Mascara, which include water, beeswax, ozokerite, shellac, glyceryl stearate, triethanolamine, propylene glycol, stearic acid, sorbitan sesquioleate, methylparaben, quaternium-15, quaternium-22, simethicone, butylparaben, and iron oxides, titanium oxides and ultramarines. These cosmetic ingredients are used in their conventional amounts and generally comprise the balance of the cosmetic products of the present invention.

The amount of cosmetic ingredients will vary widely with the type of cosmetic product, the type and amount of the other components used therein, the amount and type of POSS and non-POSS silicone resin employed and other factors well-known to those of skill in the art. However, in general, the amount of cosmetic ingredients useful in accordance with the present invention is the balance of the cosmetic product (everything other than the POSS/non-POSS Silicone Resin combination). The amount of cosmetic ingredients accordance with the present invention generally will be from about 65% to about 99.5% of the total formulation.

A person skilled in the art will take care to select the cosmetic ingredients, and the amount thereof, such that they do not harm the desired properties of the compositions, including wear and/or pliability.

Cosmetic ingredients in accordance with the present invention include, without limitation, one or more absorbents, alphahydroxy acids, betahydroxy acids, polyhydroxy acids, antiacne agents, antiperspirants, anticaking agents, antifoaming agents, antimicrobial agents, antioxidants, antidandruff agents, astringents, binders, buffers, biological additives, bulking agents, carriers, chelating agents, coupling agents, compatibilizers, conditioners, colorants, cosmetic astringents, cosmetic biocides, denaturants, drug astringents, detergents, deodorants, dispersants, external analgesics, emulsifiers, film formers, foaming agents, fragrances and fragrance components, hair styling ingredients, hair holding ingredients (mousses, sprays, etc.), hair conditioners, hair colors, hair growth promoters, humectants, keratolytics, moisturizers, straightening agents, oxidizers, mineral and organic particles, plastics, polymers, permanent waving agents, opacifying agents, perfumes, pH adjusters, pigments, preservatives, proteins, retinoids, reducing agents, sequesterants, skin bleaching agents, skin conditioning agents, skin smoothing agents, skin soothing agents, skin healing agents, softeners, solubilizing agents, solvents, surfactants, lubricants, thickeners, penetrants, permeation enhancers, analgesics, anti-inflammatory agents, antibiotics, anesthetics, plasticizers, salts, solvents essential oils, sunscreens and UV-absorbers, vitamins, provitamines, plant extracts, ceramides and pseudoceramides. The nature of the personal care ingredients used and their amounts will depend on the overall formulation, as well as its form, function and the keratinous material that is the target of its application. Understandably, the ingredients will also change depending on whether or not the product is a liquid, ointment, lotion, spray, gel, cream, liposome, microcapsule, emulsion, foam, paste, powder, granule, crystal, microcrystal or other solid. The ingredients may also depend on whether the preparations are aqueous or nonaqueous or are to be placed in a dispersion or a solution.

Cosmetic ingredients may also include, for example, keratin and its derivatives, melanin, collagen, cystine, chitosan and its derivatives, biotin, oligoelements, protein hydrolysates, and phospholipids. Other examples of cosmetic ingredients that may be mentioned are fatty alcohols, fatty acid esters of fatty alcohols, alkylated proteins, quaternized proteins, anionic, cationic, nonionic or amphoteric surfactants, silicones, volatile silicones, silicone oils, silicone gums, amino silicones, quaternized silicones, alkylated silicones, grafted silicones, silicone emulsions, mineral and plant oils, or plant waxes. Other examples of cosmetic ingredients include, without limitation, para-aminobenzoic acid (PABA), benzophenone-1, benzophenone-2, benzophenone-3, benzophenone-4, benzophenone-6, benzophenone-8, benzophenone-12, methoxycinnamate, ethyl dihydroxypropyl-PABA, glyceryl PABA, homosalate, methyl anthranilate, octocrylene, octyl dimethyl PABA, octyl methoxycinnamate, octyl salicylate, PABA, 2-phenylbenzimidazole-5-sulphonic acid, triethanolamine salicylate, 3-(4-methylbenzylidene)-camphor, avobenzone, and 2,6-dicarboxynaphtalenic acid. Other organic compounds that can be used as cosmetic ingredients include those described in U.S. Pat. No. 6,509,008 to Candau, of which the text of column 3, line 54 through column 12, line 37 are hereby incorporated by reference. Many of the cosmetic ingredients can be attached to, for example, a nitrogen containing species and can be quaternized and attached to the POSS or non-POSS silicone resin, directly or through some additional bridging group using, for example, epichlorhydrine as discussed previously.

Cosmetic products in accordance with the present invention can be made using a “cosmetically acceptable medium” made up of one or more cosmetic ingredients. Generally, these may be composed of a fatty phase, optionally organic solvents and optionally water or other solvents in amounts such that they do not interfere with the improved wear and or flexibility imparted by the POSS/non-POSS mixture.

The fatty phase may be made of generally fatty substances that are liquid at room temperature (generally 25° C.) and/or of fatty substances that are solid at room temperature, such as waxes, gums and pasty fatty substances, and mixtures thereof.

As fatty substances that are liquid at room temperature, often referred to as oils, which may be used in the invention, mention may be made of silicone oils, hydrocarbon-based oils, of mineral, animal, plant or synthetic origin, alone or as a mixture provided that they form a homogeneous and stable mixture and provided that they are compatible with the intended use.

The cosmetically acceptable medium preferably contains volatile and/or non-volatile silicone oils.

Non-volatile silicone oils which may be mentioned are polydimethylsiloxanes (PDMSs), that are optionally phenylated, such as phenyltrimethicones, phenyltrimethylsiloxydiphenylsiloxanes, diphenylmethyldimethyltrisiloxanes, diphenyldimethicones, phenyldimethicones and polymethylphenylsiloxanes, optionally substituted with aliphatic and/or aromatic groups, or optionally fluorinated; polysiloxanes modified with fatty acids, fatty alcohols or polyoxyalkylenes, fluorosilicones and perfluorosilicone oils, and mixtures thereof.

Among the non-volatile silicone oils that are useful, mention may be made of polydimethylsiloxanes, polymethylphenylsiloxanes, silicones comprising polyoxyalkylene blocks or grafts, in particular polyoxyethylene or copoly(oxyethylene/oxypropylene) blocks or grafts, such as dimethicone copolyols, silicones bearing both hydrophobic hydrocarbon-based groups (for example C₂-C₃₀ alkyl groups) and polyoxyethylenated or copoly(oxyethylenated/oxypropylenated) blocks or grafts, such as alkyldimethicone copolyols, silicones bearing fluoro or perfluoro groups such as perfluoroalkyl polydimethylsiloxanes and perfluoroalkyl polymethylphenylsiloxanes, and mixtures thereof.

One or more oils that are volatile at room temperature may also be used. These volatile oils make it easier to apply the composition to the skin, the lips and superficial body growths.

The term “volatile oil” means an oil which is capable of evaporating at the temperature of the skin or the lips, and which has a non-zero vapour pressure at room temperature and under atmospheric pressure, ranging in particular from 0.13 to 4.0×10⁴ Pa (10-3 to 300 mm Hg) and better still greater than 40 Pa (0.3 mm Hg).

These oils may be silicone oils optionally comprising alkyl or alkoxy groups at the end of or pendent on a silicone chain.

As volatile silicone oils which may be used in the invention, mention may be made of linear or cyclic silicones with a viscosity at room temperature and under atmospheric pressure of less than 8 mm2/s (8 cSt) and in particular comprising from 2 to 7 silicon atoms. Mention may be made in particular of octamethylcyclotetrasiloxane, decamethylcyclopentasiloxane, hexadecamethylcyclohexasiloxane, heptamethylhexyltrisiloxane and heptamethyloctyltrisiloxane, and mixtures thereof.

Preferably, at least one volatile silicone oil chosen especially from octamethylcyclotetrasiloxane, decamethylcyclopentasiloxane, hexadecamethylcyclohexasiloxane, heptamethylhexyltrisiloxane, heptamethyloctyltrisiloxane, octamethyltrisiloxaneand decamethyltetrasiloxane, and mixtures thereof, may be used.

The cosmetically acceptable medium may also contain one or more oils of non-silicone nature such as, for example, hydrocarbon-based oils.

The term “hydrocarbon-based oil” means an oil predominantly containing carbon and hydrogen atoms, and in particular alkyl or alkenyl chains such as alkanes or alkenes, as well as an oil not only containing hydrogen and carbon atoms, but also oxygen atoms, in the form of an ether, ester, alcohol or carboxylic acid function.

Mention may also be made of hydrocarbon-based oils such as liquid paraffin or liquid petroleum jelly, mink oil, turtle oil, soyabean oil, perhydrosqualene, sweet almond oil, beauty-leaf oil, palm oil, grape pip oil, sesame oil, corn oil, parleam oil, arara oil, rapeseed oil, sunflower oil, cottonseed oil, apricot oil, castor oil, avocado oil, jojoba oil, olive oil or cereal germ oil; esters of linoleic acid, of oleic acid, of lauric acid or of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, bis(2-ethylhexyl) succinate, diisostearyl malate and glyceryl or diglyceryl triisostearate; higher fatty alcohols containing at least 12 carbon atoms, such as stearyl alcohol, oleyl alcohol, linoleyl alcohol, linolenyl alcohol, isostearyl alcohol or octyldodecanol.

For the purposes of the present invention, a wax is a lipophilic compound which is solid at room temperature (about 25° C.), which undergoes a reversible solid/liquid change of state, which has a melting point above about 40° C., which may be up to 200° C., and which has an anisotropic crystal organization in the solid state. In general, the size of the wax crystals is such that the crystals scatter and/or diffuse light, giving the composition comprising them a more or less opaque cloudy appearance. By bringing the wax to its melting point, it is possible to make it miscible with oils and to form a microscopically homogeneous mixture, but on returning the temperature of the mixture to room temperature, a recrystallization of the wax in the oils of the mixture is obtained, which may be detected microscopically and macroscopically (opalescence).

As examples of waxes which may be used according to the invention, mention may be made of waxes of animal origin such as beeswax, spermaceti, lanolin wax and lanolin derivatives; plant waxes such as carnauba wax, candelilla wax, ouricury wax, Japan wax, cocoa butter, cork fibre wax of sugar cane wax; mineral waxes, for example paraffin wax, petroleum jelly wax, lignite wax, microcrystalline waxes or ozokerites; synthetic waxes including polyethylene wax, polytetrafluoroethylene wax and the waxes obtained by Fisher-Tropsch synthesis, or alternatively silicone waxes, hydrogenated oils that are solid at 25° C., such as hydrogenated castor oil, hydrogenated jojoba oil, hydrogenated palm oil, hydrogenated tallow and hydrogenated coconut oil, and fatty esters that are solid at 25° C., such as the C₂₀-C₄₀ alkyl stearate sold under the trade name Kester Wax K82H by the company Koster Keunen.

The gums are generally high molecular weight polydimethylsiloxanes (PDMSs) and the pasty substances are generally hydrocarbon-based compounds, for instance lanolins and derivatives thereof, or PDMSs.

The cosmetic compositions according to the invention may include one or more cosmetically acceptable (acceptable tolerance, toxicology and feel) organic solvents. These organic solvents may be chosen from hydrophilic organic solvents, lipophilic organic solvents and amphiphilic solvents, and mixtures thereof.

Among the hydrophilic organic solvents which may be mentioned, for example, are linear or branched lower monoalcohols containing from 1 to 8 carbon atoms, for instance ethanol, propanol, butanol, isopropanol, isobutanol; acetone; polyethylene glycols containing from 6 to 80 ethylenoxy units; polyols such as propylene glycol, butylene glycol, glycerol or sorbitol; esters such as ethylacetate and methylacetate, mono- or dialkyl isosorbides in which the alkyl groups contain from 1 to 5 carbon atoms, for instance, dimethyl isosorbide; for instance, diethylene glycol monomethyl ether or monoethyl ether and propylene glycol ethers, such as dipropylene glycol methyl ether.

Other organic solvents which may be mentioned include polyols such as polypropylene glycol (PPG) derivatives, for instance polypropylene glycol esters of fatty acids, and PPG ethers of fatty alcohols, for example PPG-36 oleate and PPG-23 oleyl ether.

Lipophilic organic solvents which may be mentioned, for example, include hydrocarbons such as hexane, heptane and octane; fatty esters such as diisopropyl adipate and dioctyl adipate; alkyl benzoates; and dioctyl malate.

Fillers may also be used, including mineral or organic, and lamellar or spherical fillers. Mention may be made of talc, mica, silica, kaolin, Nylon® (orgasol® from Atochem) powder, polyalanine powder and polyethylene powder, Teflon®, lauroyllysine, starch, boron nitride, hollow microspheres such as Expancel® (Nobel Industry), Polytrap® (Dow Corning) and silicone resin microbeads (Tospearls® from Toshiba, for example), precipitated calcium carbonate, magnesium carbonate and magnesium hydrocarbonate, hydroxyapatite, hollow silica microspheres (silica beads from Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate.

“Colorants” or “coloring agents” useful in the cosmetic compositions of the invention may include, for example, pigments, including nacreous pigments, solid particles (for example, glitter flakes) and lipsoluble colorants. Pigments may be white or coloured, and mineral and/or organic. Among the mineral pigments which may be mentioned are titanium dioxide, optionally surface-treated, zirconium oxide or cerium oxide, and iron oxide or chromium oxide, manganese violet, ultramarine blue, chromium hydrate and ferric blue. Among the organic pigments which may be mentioned are carbon black, pigments of D&C type, and lakes based on cochineal carmine or on barium, strontium, calcium or aluminum.

Nacreous pigments may be used and may be chosen from white nacreous pigments such as mica coated with titanium oxide or with bismuth oxychloride, coloured nacreous pigments such as titanium mica with iron oxides, titanium mica with, in particular, ferric blue or chromium oxide, titanium mica with an organic pigment of the above-mentioned type and nacreous pigments based on bismuth oxychloride.

Liposoluble colorants may be used and include, for example, Sudan Red, DC Red 17, DC Green 6, β-carotene, DC Yellow 11 or DC Violet 2. They may represent from 0.0% to 20% of the weight of the composition and better still from 0.1% to 6%.

One example of a cosmetic composition of the invention is a nail varnish. The nail varnish may include nitrocellulose, cellulose acetopropionates and cellulose ester. The acetopropionates may be present in an amount ranging from 10% to 80% by weight, relative to the total weight of cellulose ester and nitrocellulose present in the composition. In another embodiment of the invention, cellulose acetopropionates can be present in the nail varnish composition in an amount ranging from 12.5% to 75% by weight, relative to the total weight of cellulose ester and nitrocellulose present in the composition.

Nitrocellulose can be present in the nail varnish composition in an amount ranging from 1.5 to 35% by weight relative to the total weight of the composition. In another embodiment, the nitrocellulose can be present in the composition in an amount ranging from 8% to 20% by weight, relative to the total weight of the composition. Nitrocellulose and cellulose esters useful in accordance with the present invention are identified in U.S. Pat. No. 6,333,025 to Ramin, the text of which is hereby incorporated by reference.

According to another embodiment of the invention, the composition comprises at least one film-forming polymer in addition to nitrocellulose and cellulose ester, at least to improve the cosmetic and physicochemical properties of a nail varnish film.

In yet another embodiment of the invention, the additional film-forming polymer can be present in the nail varnish composition in an amount up to 50% by weight, and is preferably present in an amount less than 40% by weight, relative to the total weight of nitrocellulose and cellulose ester. In still another embodiment of the invention, the amount of additional film forming polymer ranges from 1% to 15% by weight relative to the total weight of nitrocellulose and cellulose ester. These film-forming polymers are in addition to the POSS and non-POSS silicone resin mixture.

The solvent for the nail varnish composition can comprise at least one organic solvent, examples of which include:
ketones which are liquid at room temperature, such as methyl ethyl ketone, methyl isobutyl ketone, diisobutyl ketone, isophorone, cyclohexanone and acetone;
alcohols which are liquid at room temperature, such as ethanol, isopropanol, diacetone alcohol, 2-butoxyethanol and cyclohexanol;
glycols which are liquid at room temperature, such as ethylene glycol, propylene glycol, pentylene glycol and glycerol;
propylene glycol ethers which are liquid at room temperature, such as propylene glycol monomethyl ether, propylene glycol monomethyl ether acetate and dipropylene glycol mono-n-butyl ether;
short-chain esters (containing from 3 to 8 carbon atoms in total), such as ethyl acetate, methyl acetate, propyl acetate, n-butyl acetate and isopentyl acetate;
ethers which are liquid at room temperature, such as diethyl ether, dimethyl ether and dichlorodiethyl ether;
alkanes which are liquid at room temperature, such as decane, heptane, dodecane and cyclohexane;
aromatic cyclic compounds which are liquid at room temperature, such as toluene and xylene;
aldehydes which are liquid at room temperature, such as benzaldehyde and acetaldehyde, and mixtures thereof.

Nail varnish compositions according to the invention can further comprise water in an amount ranging up to 90% by weight, relative to the total weight of the composition.

Nail varnish compositions according to the invention may also comprise at least one plasticizer. Examples of plasticizers suitable for use in the present invention, alone or as a mixture, include:
glycols and derivatives thereof such as diethylene glycol ethyl ether, diethylene glycol methyl ether, diethylene glycol butyl ether, diethylene glycol hexyl ether, ethylene glycol ethyl ether, ethylene glycol butyl ether and ethylene glycol hexyl ether;
glycerol esters;
propylene glycol derivatives including propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether;
acid esters, including carboxylic acid esters, such as citrates, phthalates, adipates, carbonates, tartrates, phosphates and sebacates;
oxyethylenated derivatives, including oxyethylenated oils, for example, plant oils such as castor oil; and mixtures thereof.

The nail varnish can also include an amount of plasticizer, which can be chosen by a person skilled in the art on the basis of his or her general knowledge, so as to obtain a composition which has cosmetically acceptable properties. The amount of plasticizer present in the composition can range, for example, from 0.5% to 20% by weight relative to the total weight of the composition. In one embodiment of the invention, the amount of plasticizer ranges from 2% to 10% by weight relative to the total weight of the composition.

The colorant, such as Dyestuff present in the composition can be chosen from pulverulent compounds and dyes which are soluble in the solvent medium of the composition, in an amount which is sufficient to dye the varnish in a color in the visible wavelength range, i.e., between 400 and 800 nm, and, for example, in a content ranging from 0.001% to 10% by weight relative to the total weight of the composition. The pulverulent compounds can be chosen from the pigments, nacres and glitter flakes usually used in nail varnishes.

The glitter flakes can be chosen from those made of acrylic, of polyester and of polyethylene terephthalate, and of aluminum.

The dyes can be, for example, Sudan red, DC Red 17, DC Green 6, α-carotene, soybean oil, Sudan brown, DC Yellow 11, DC Violet 2, DC Orange 5 and cannelloni yellow.

Nail varnish compositions of the invention can also optionally comprise an optical brightener, for example to reduce the yellowing effect of the nitrocellulose and consequently of the composition, this brightener not being considered, for the purposes of the invention, as a dyestuff. One brightener which can be used is the monosodium salt of alizurol purple; this brightener can be present in the inventive compositions in an amount ranging from 10-7% to 10-4% by weight relative to the total weight of the composition.

Nail varnish compositions according to the invention can also comprise any cosmetic ingredient known to those skilled in the art capable of being incorporated into dye compositions. Such ingredients include thickeners, spreading agents, wetting agents, dispersing agents, antifoaming agents, preserving agents, UV screening agents, active agents, surfactants, moisturizers, fragrances, neutralizers, stabilizers, and antioxidants. Needless to say, a person skilled in the art will take care to select one or more optional cosmetic ingredients, and/or the amount thereof, such that the advantageous properties of the POSS/non-POSS composition are not, or are not substantially, adversely affected by the additional ingredients. In an embodiment of the invention, the composition is free or essentially free of silicone drying agents. See also U.S. Pat. No. 6,372,201 to Leuridan et al., the text of which is also incorporated by reference. The amount of POSS used in any nail polish may vary. Preferably, however, the amount ranges from about 0.005 to about 40% w/w. More preferably about 0.05 to about 25% (w/w). Even more preferably about 0.1 to about 20% (w/w). These same amounts may be used in other nail products as polishes and enamels as well and indeed most personal care products.

Indeed, the POSS/non-POSS mixture may be included in any of the nail varnish compositions of U.S. Pat. No. 6,333,025, and any other known nail polish, varnish or enamel. Nail enamels that may be used in accordance with the present invention include those disclosed in U.S. Pat. No. 6,555,096.

Another example of a cosmetic composition in accordance with the invention is a nail enamel. Nail enamels typically contain, in an organic solvent or mixture of solvents, film-forming ingredients, plasticizing ingredients, and colorants. Of course, the film-forming ingredients can be the POSS and/or non-POSS silicone resin, and does not have to be an additional substance. Generally, the composition also contains a thixotropic agent, which may act to thicken the composition in order to allow better spreading on the nail. The thixotropic agent may also act to suspend the colorant.

The classic thixotropic agent used in the prior art is a bentonite clay. Aromatic organic solvents in particular may cause these clays to swell, thus providing a gel with good thixotropic properties, i.e., rendering the composition capable of passing from a gelled state to a liquid state simply by stirring and capable of going from liquid to gel after standing. A composition containing such a gel thus may exhibit relatively good dispersion stability without sedimentation or separation over a long Period. Further, such compositions may not require the vigorous shaking that other compositions often require after extended periods of storage. Urea-modified thixotropic agents made from modified urethanes are disclosed in U.S. Pat. No. 6,555,096 to Carrion, the text of which is hereby incorporated by reference.

Nail enamel compositions preferably include a cosmetically acceptable solvent system comprising diacetone alcohol and at least one additional solvent chosen from C₁-C₆ alkyl acetates and C₁-C₆ alkyl alcohols; at least one film-forming substance; and at least one thixotropic agent. The nail enamel composition of the invention may also contain a plasticizer and optionally a coloring agent as described for varnishes.

The nail enamel composition of the invention may contain at least one additional thixotropic agent, used in conjunction with the at least one urea-modified thixotropic agent. When such an additional thixotropic agent is present, the composition may comprise from about 0.10 to about 0.30% of the at least one urea-modified thixotropic agent relative to the weight of the composition and up to about 1.0% of the additional thixotropic agent. The additional thixotropic agent(s) may be chosen from conventional silica and bentonite clay agents. It is also possible to use these additional thixotropic agents instead of urea-modified agents as desired.

Film forming substances (other than the POSS/non-POSS resins) useful in the present invention include, but are not limited to, conventional film-forming agents such as nitrocellulose, other cellulose derivatives, such as cellulose acetate, cellulose acetate butyrate, and ethyl cellulose; polyesters; resins, such as polyurethane resins, alkyd resins, and polyvinyl resins such as polyvinyl acetate, polyvinyl chloride, polyvinylbutyrate; (meth)acrylic and vinyl copolymers such as styrene/butadiene copolymers, acrylate/vinyl acetate copolymers, acrylonitrile/butadiene copolymers, and ethylene/vinyl acetate copolymers.

In one embodiment, the primary film-forming agent may be nitrocellulose, which is known to give hardness and resistance to abrasion. If a second film-forming substance is present, this second film-forming substance may, for example, be chosen from cellulose acetate butyrate and acrylates. In one embodiment, the film-forming substance may be present in an amount of from about 5% to about 20% by weight relative to the weight of the composition, for example, from about 10% to about 14%. Suitable modifiers for the primary film-forming agent include arylsulfonamide resins such as arylsulfonamide formaldehyde or epoxy resins.

The presently disclosed nail enamel composition including the POSS/non-POSS composition also may contain at least one plasticizer. Plasticizers useful in the presently disclosed nail enamel composition include plasticizers commonly employed in nail varnish compositions. These plasticizers encompass, but are not limited to, dibutyl phthalate, dioctyl phthalate, tricresyl phthalate, butyl phthalate, dibutoxy ethyl phthalate, diamylphthalate, tosyl amide, N-ethyl-tosyl amide, sucrose acetate isobutyrate, camphor, castor oil, citrate esters, glyceryl diesters, glyceryl triesters, tributyl phosphate, tri-phenyl phosphate, butyl glycolate, benzyl benzoate, butyl acetyl ricinoleate, butyl stearate, and dibutyl tartrate. In one embodiment, a plasticizer used in the present invention may be the mixture of acetyl tributyl citrate and N-ethyl tosyl amide. The plasticizer may, for example, be present in an amount of from about 3% to about 12% by weight relative to the weight of the composition.

One solvent for a nail enamel of the present invention comprises diacetone alcohol and at least one additional solvent chosen from C₁-C₆ alkyl acetates and C₁-C₆ alkyl alcohols. In one embodiment, the C₁-C₆ alkyl acetates are chosen from ethyl acetate, propyl acetate, and butyl acetate. In another embodiment, the C₁-C₆ alkyl alcohols are chosen from ethanol, isopropanol, and butanol. Other cosmetically acceptable organic solvents which can be used in conjunction with the inventive solvent system include, but are not limited to, toluene; xylene; ketones such as acetone or methyl ethyl ketone; glycol ethers; alkanes such as hexane or heptane; -methylpyrrolidone; and alkyl lactates. The solvent system of the invention, including any additional solvents, may be present, in one embodiment, in an amount of from about 40% to about 80% by weight relative to the weight of the composition, and, in another embodiment, from about 65% to about 78%.

The nail enamel composition of the invention may also contain at least one coloring agent. Conventional coloring agents can be used, and examples include inorganic pigments such as titanium dioxide, iron oxides, titanated mica, iron oxide coated mica, ultramarine, chromium oxide, chromium hydroxide, manganese violet, bismuth oxychloride, guanine, and aluminum; pearlescent materials; and organic coloring agents such as ferric ammonium ferrocyanide, and D&C Red Nos. 6, 7, 34; Blue No. 1; Violet No. 2; and Yellow No. 5.

The colorant can also include one or more pigments. These pigments can be white or colored, and inorganic or organic. Examples of inorganic pigments include titanium dioxide, which has optionally been surface-treated, zirconium oxide and cerium oxide, as well as iron oxide and chromium oxide, manganese violet, ultramarine blue, chromium hydrate and ferric blue, and metallic pigments such as aluminum and bronze. Examples of organic pigments include carbon black, pigments of D&C type and lakes based on cochineal carmine, barium, strontium, calcium, aluminum, and guanine.

The nacreous pigments can be chosen from white nacreous pigments such as mica coated with titanium or with bismuth oxychloride, colored nacreous pigments such as titanium mica with, for example, iron oxides, ferric blue, chromium oxide, or with an organic pigment of the above-mentioned type, as well as nacreous pigments based on bismuth oxychloride.

The inorganic pigments may be surface-treated as is customary to prevent migration or striation. Silicones and polyethylenes are most often used as the coatings for inorganic pigments and thus may be used according to the present invention. Colorant materials may also include chips or powder of mica or diamonds in the nail composition. Also useful are specialty materials giving rise to two-tone color effects such as liquid crystal silicones or multi-lamellar metallic particulates, which generally can be mixed with pigments or dyes to obtain a broader spectrum of brilliant color and increased luminous reflectance. Such materials are described in, e.g., U.S. Pat. Nos. 3,438,796; 4,410,570; 4,434,010; 4,838,648; 4,930,866; 5,171,363; 5,364,467; 5,569,535; 5,607,904; 5,624,486; 5,658,976; 5,688,494; 5,766,335; N. Hatberle et al., “Right and Left Circular Polarizing Colorfilters made from Crosslinkable Cholesteric LC-Silicones,” Conference Record of the 1991 International Display Research Conference (IEEE), pp. 57-59; R. Maurer et al., “Polarizing Color Filters made from Cholesteric LC-Silicones,” SID 90 Digest (1990), pp. 110-113; H.-J. Eberle et al., “Inverse Angle Dependence of the Reflection Colours of Cholesteric Polymeric Liquid Crystals Mixed with Pigments,” Liquid Crystals, 5(3), (1989), pp. 907-916; J. Pinsl et al., “Liquid Crystalline Polysiloxanes for Optical Once-Write Storage,” J. Molec. Electr., Vol. 3 (1987), pp. 9-13; and D. Makow, “Reflection and Transmission of Polymer Liquid-Crystal Coatings and their Application to Decorative Arts and Stained Glass,” Color Res. Applic. Vol. 11, No. 3, (1986), pp. 205-208, all of which are incorporated herein by reference in their entirety.

In one embodiment, the colorant may be present in the nail enamel composition in an amount up to about 5% by weight relative to the total weight of the composition. In another embodiment, the colorant is present in an amount of from 2% to 3% by weight.

POSS molecules particularly useful in a composition for nail care products include, without limitation:

MERCAPTOPROPYLISOOCTYL-POSS (TH1555)

Epoxides POSS:

EpoxyCyclohexylCyclohexyl-POSS (EP0399)

EpoxyCyclohexylCyclopentyl-POSS (EP0400)

EpoxyCyclohexylIsobutyl-POSS (EP0402)

EpoxyCyclohexyl-POSS Cage Mixture (EP0408)

GlycidylIsooctyl-POSS (EP0419)

OctaGlycidyldimethylsilyl-POSS (EP0435), etc.

There are a number of specific formulations that may produce the desired improvement in wear and/or pliability. Examples of these formulations include, without limitation:

### EXAMPLE 1

### Lip Paint Stick Base Coat

Lip paint stick base coat, examples A, B and C, were prepared by mixing the following ingredients:

| Trade Name | INCI Name | A | B | C |
|---|---|---|---|---|
| Permethyl 99A | Isododecane | 10.00 | 20.00 | 20.00 |
| DC 200 fluid | Dimethicone | 20.39 | 20.39 | 20.39 |
| MS0805 | Isooctyl-POSS | 5.00 | 5.00 | 5.00 |
| DC 200 fluid, 60,000 cst | Dimethicone | 6.00 | 5.00 | |
| Eastman SAIB | Sucrose Acetate | | | 4.50 |
| | Isobutyrate | | | |
| SF 1642 | C30-45 Alkyl | 9.0 | 9.0 | 5.0 |
| | Dimethicone | | | |
| Polywax 655 | Polyethylene | 10.0 | 11.0 | 15.0 |
| Black Iron Oxide | Iron Oxides | 0.24 | 0.24 | 0.24 |
| FDC Red 21 Al Lake | Red 21 Lake | 1.83 | 1.83 | 1.83 |
| DC Red 7 W | Red 7 Lake | 0.24 | 0.24 | 0.24 |
| Yellow Iron Oxide | Iron Oxides | 0.97 | 0.97 | 0.97 |
| Red Iron Oxide | Iron Oxides | 1.83 | 1.83 | 1.83 |
| DUB VCI 10 | Isodecyl | 10.0 | 10.0 | 10.0 |
| | neopentanoate | | | |
| Abil wax 9814 | Cetyl dimethicone | 1.0 | 1.0 | 1.0 |
| Color Grind 479-168-1 | | 16.11 | 16.11 | 16.11 |
| Flamenco Superpearl | Mica & TiO₂ | 1.50 | 1.50 | 3.00 |
| 120 C | | | | |
| MSS-500/3H | Silica | 1.00 | 1.00 | 1.00 |
| Actives | | 1.00 | 1.00 | |
| Permethyl 99A | Isododecane | 10.0 | 10.0 | 10.0 |
| Total: | | 100.0 | 100.0 | 100.0 |

### EXAMPLE 2

Silicone type lipsticks, A-F, were prepared according to the following formulation:

| Trade Name | INCI Name | A | B | C | D | E | F |
|---|---|---|---|---|---|---|---|
| MS0805 | Isooctyl-POSS | | 10.00 | | | | |
| MS0830 | Octamethyl-POSS | | | | | 10.00 | |
| MS0825 | Octaisobutyl-POSS | | | | | | 10.00 |
| MS0840 | Octaphenyl-POSS | | | 10.00 | 10.00 | | |
| DC 200 | Dimethicone | 10.00 | | | | | |
| fluid, | | | | | | | |
| 60,000 cst | | | | | | | |
| Schercemol | Triethylhexanoin | 18.69 | 18.69 | 18.69 | | | |
| GTO | | | | | | | |
| Tegosoft | Diethylhexyl | | | | 18.69 | 18.69 | 18.69 |
| DEC | carbonate | | | | | | |
| Dragoxate | Ethylhexyl | 10.00 | 10.00 | 10.00 | 10.00 | 10.00 | 10.00 |
| EH | ethylhexanoate | | | | | | |
| SR 1000 | Trimethyl- | 10.00 | 10.00 | 10.00 | 10.00 | 10.00 | 10.00 |
| | syloxysilicate | | | | | | |
| BHT | BHT | 0.05 | 0.05 | 0.05 | 0.05 | 0.05 | 0.05 |
| SF1642 | C30-45 alkyl | 9.0 | 9.00 | 9.0 | 9.0 | 9.0 | 9.0 |
| | Dimethicone | | | | | | |
| Polyethylene | Polyethylene | 9.50 | 9.50 | 9.50 | 9.50 | 9.50 | 9.50 |
| 500 | | | | | | | |
| Titanium | Titanium Dioxide | 0.75 | 0.75 | 0.75 | 0.75 | 0.75 | 0.75 |
| Dioxide | | | | | | | |
| Yellow 6 | Yellow 6 | 1.35 | 1.35 | 1.35 | 1.35 | 1.35 | 1.35 |
| Blue 1 | Blue 1 | 0.25 | 0.25 | 0.25 | 0.25 | 0.25 | 0.25 |
| Red 7 | Red 7 Lake | 0.30 | 0.30 | 0.30 | 0.30 | 0.30 | 0.30 |
| Red Iron | Iron Oxides | 0.81 | 0.81 | 0.81 | 0.81 | 0.81 | 0.81 |
| Oxide | | | | | | | |
| Coslin C- | Kaolin | 5.00 | 5.00 | 5.00 | 5.00 | 5.00 | 5.00 |
| 100 | | | | | | | |
| Dragoxate | Ethylhexanoate | 18.00 | 18.00 | 18.00 | 18.00 | 18.00 | 18.00 |
| EH | | | | | | | |
| Abil Wax | Cetyl Diemthicone | 1.0 | 1.0 | 1.0 | 1.0 | 1.0 | 1.0 |
| 9814 | | | | | | | |
| | Color Grind 527-55-2 | 27.46 | 27.46 | 27.46 | 27.46 | 27.46 | 27.46 |
| Timiron | Mica & TiO₂ | 3.50 | 3.50 | 3.50 | 3.50 | 3.50 | 3.50 |
| Super Silk | | | | | | | |
| MP 1005 | | | | | | | |
| MSS-500/3H | Silica | 0.80 | 0.80 | 0.80 | 0.80 | 0.80 | 0.80 |
| Actives | | 1.0 | 1.0 | 1.0 | 1.0 | 1.0 | 1.0 |
| Total: | | 100 | 100 | 100 | 100 | 100 | 100 |

### EXAMPLE 3

Comparison of formula A & B of Example 2, above: after applying formula A onto the lips, the lipstick film is brittle and easily flakes off, while after applying formula B onto the lips, the lipstick film is flexible and lasted up to 8 hours.

### EXAMPLE 4

Long-Lasting Foundation

| | % w/w |
|---|---|
| Oil Phase | |
| Emulsifiers | 2.50 |
| Volatile oils | 29.47 |
| Silicone resin | 8.00 |
| Isooctyl POSS | 5.50 |
| Pigments and fillers | 14.00 |
| Rheological | 1.00 |
| additives/preservatives | |
| Aqueous Phase | |
| Deionized Water | 32.23 |
| Propylene GLYCOL | 5.00 |
| SODIUM CHLORIDE | 2.00 |
| Preservatives | 0.30 |
| Total | 100.00 |

### EXAMPLE 5

Long-Lasting Mascara

| | % w/w |
|---|---|
| Carnauba wax | 7.00 |
| Beeswax | 2.00 |
| Microcrystalline was | 20.00 |
| Isododecane | 49.50 |
| Silicone resin | 10.00 |
| Isoocytl Pass | 5.50 |
| Pigments and fillers | 10.00 |
| Bentonite | 3.00 |
| Total | 100.00 |

### EXAMPLE 6

Long-Lasting Liquid Eye Liner

| | % w/w |
|---|---|
| Water | 50-70 |
| Gellant | 0.5-1.5 |
| Wetting agents | 1-3 |
| Polyol | 4-8 |
| Silicone resin | 3-10 |
| Isooctyl POSS | 3-10 |
| Colorants | 10-20 |

1. A film forming composition comprising: a polyhedral oligomeric silsesquioxane and non-polyhedral oligomeric silsesquioxane silicone resin, wherein said non-polyhedral oligomeric silsesquioxane silicone resin is selected from the group consisting of a siloxysilicate or a polysiloxane and wherein the ratio of polyhedral oligomeric silsesquioxane to the non-polyhedral oligomeric silsesquioxane silicone resin can range from about 1:4 to about 1:1.

2. The film forming composition of claim 1 wherein said siloxysilicate has the formula [R₃—Si—O]x-(SiO_{4/2})y, wherein x and y range from about 50 to 80, and said polysiloxane has the formula [R₃—Si—O]—(R₂SiO)ₓ—[Si—R₃], wherein x is at least 2000 and for siloxysilicates and polysiloxanes said R groups is an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin group.

3. The film forming composition of claim 1 wherein said ratio of polyhedral oligomeric silsesquioxane to the non-polyhedral oligomeric silsesquioxane silicone resin can range from about 1:3 to about 9:11.

4. The film forming composition of claim 1 wherein said polyhedral oligomeric silsesquioxane is an isooctyl polyhedral oligomeric silsesquioxane and said non-polyhedral oligomeric silsesquioxane silicone resin is dimethicone.

5. The film forming composition of claim 1 wherein said polyhedral oligomeric silsesquioxane includes at least one M, D or T subunit having at least one R group selected from the group consisting of a hydrogen, methyl, ethyl, propyl, isobutyl, isooctyl, phenyl, cyclohexyl, cyclopentyl, —OSi (CH₃)₂—CH₂—CH₂—(CF₂)₅CF₃, —(CH₂)₃SH, N⁺(CH₃)₃, O⁻N⁺(CH₃)₃, —OH, —(CH₂)ₙN⁺H₃X⁻ wherein n is 0-30 and X is a counter ion,

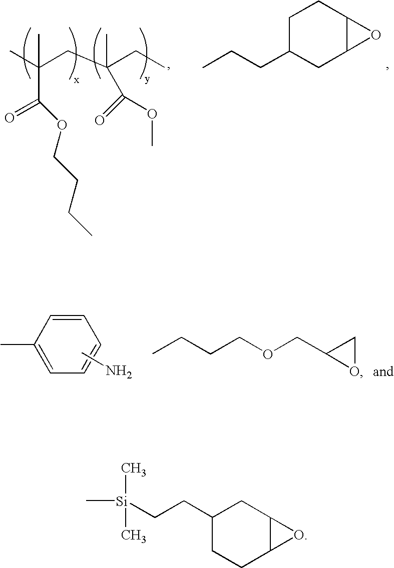

6. A film forming composition comprising: a combination of a polyhedral oligomeric silsesquioxane having a three dimensional cage structure formed of a plurality of Si subunits, at least one of said subunits including at least one R group, and a non-polyhedral oligomeric silsesquioxane silicone resin with the proviso that if said non-polyhedral oligomeric silsesquioxane is a polymethylsilsesquioxane, then said polyhedral oligomeric silsesquioxane will have other than 8 Si subunits, or at least one of the R groups of the polyhedral oligomeric silsesquioxane will be other than a methyl group or said polyhedral oligomeric silsesquioxane will have an incomplete cage structure.

7. The film forming composition of claim 6, wherein the ratio of polyhedral oligomeric silsesquioxane to the non-polyhedral oligomeric silsesquioxane silicone resin will range from about 1:4 to about 1:1.

8. The film forming composition of claim 6, wherein the ratio of polyhedral oligomeric silsesquioxane to the non-polyhedral oligomeric silsesquioxane silicone resin will range from about 1:3 to about 9:11.

9. A cosmetic composition comprising: a polyhedral oligomeric silsesquioxane formed of a plurality of Si subunits, at least one of the subunits having one or more R groups, a non-polyhedral oligomeric silsesquioxane silicone resin, a solvent, a coloring agent, and at least one additional cosmetic ingredient, wherein the combined amount of polyhedral oligomeric silsesquioxane and non-polyhedral oligomeric silsesquioxane silicone resin will range from about 0.5% to about 35% by weight of said cosmetic composition.

10. The cosmetic composition of claim 9 wherein if said silicone resin is a polymethylsilsesquioxane, said polyhedral oligomeric silsesquioxane will have other than 8 Si subunits, or at least one of said R groups is something other than a methyl group, or said polyhedral oligomeric silsesquioxane will have an incomplete cage structure.

11. The cosmetic composition of claim 9 wherein said ratio of polyhedral oligomeric silsesquioxane to the non-polyhedral oligomeric silsesquioxane silicone resin will range from about 1:4 to about 1:1.

12. The cosmetic composition of claim 11 wherein said ratio of polyhedral oligomeric silsesquioxane to the non-polyhedral oligomeric silsesquioxane silicone resin will range from about 1:3 to about 9:11.

13. The cosmetic composition of claim 9 wherein said non-polyhedral oligomeric silsesquioxane silicone resin is a Q resin.

14. The cosmetic composition of claim 13 wherein said non-polyhedral oligomeric silsesquioxane silicone resin is selected from the group consisting or a siloxysilicate or a polysiloxane, said siloxysilicate having the formula [R₃—Si—O]x-(SiO_{4/2})y, wherein x and y range from about 50 to 80, and said polysiloxane having the formula [R₃—Si—O]—(R₂SiO)ₓ—[Si—R₃], wherein x is at least 2000 and for siloxysilicates and polysiloxanes, said R2 and R3 groups, which may be the same or different, and are an alkyl, hydroxyl, alkoxysilane, amine, chlorosilane, epoxide, ester, halide, methacrylate, molecular silica, nitrile, norborene, olefin, phosphine, silane, silanol, styrenic polymer, or polyolefin group.

15. The cosmetic composition of claim 13 wherein said non-polyhedral oligomeric silsesquioxane silicone resin comprises dimethicone or trimethylsyloxysilicate.

16. (canceled)

17. The cosmetic composition of claim 13 wherein said polyhedral oligomeric silsesquioxane is an isooctyl polyhedral oligomeric silsesquioxane.

18. The cosmetic composition of claim 13 which is a foundation, mascara, eye liner, concealer, lip coating, blush, body make-up, and nail coating.

19. The cosmetic composition of claim 13 wherein said additional cosmetic ingredient is an absorbent, alphahydroxy acid, betahydroxy acid, polyhydroxy acid, antiacne agent, anticaking agent, antifoaming agent, antimicrobial agent, antioxidant, astringent, binder, bleaching agent, buffer, biological additive, bulking agent, carrier, chelating agent, coupling agent, compatibilizer, conditioner, colorant, cosmetic astringent, cosmetic biocide, denaturant, drug astringent, detergent, deodorant, dispersant, external analgesic, emulsifier, film former, foaming agent, fragrance and fragrance component, humectant, keratolytic, moisturizer, straightening agent, oxidizer, mineral and organic particle, plastic, polymer, opacifying agent, perfume, pH adjuster, preservative, protein, retinoid, reducing agent, sequesterant, skin bleaching agent, skin conditioning agent, skin smoothing agent, skin soothing agent, skin healing agent, softener, solubilizing agent, surfactant, lubricant, thickener, penetrant, permeation enhancer, analgesic, anti-inflammatory agent, antibiotic, anesthetic, plasticizer, salt, solvent essential oil, sunscreen and UV-absorber, vitamin, provitamine, plant extract, ceramide or pseudoceramide.

20. The cosmetic composition of claim 13 provided in the form of a liquid, solid, cream, ointment, solution, gel, mousse, stick, spray, powder, emulsion or dispersion.

21. A cosmetic composition comprising:
an iso-octyl polyhedral oligomeric silsesquioxane;
a siloxysilicate, wherein the ratio of said iso-octyl polyhedral oligomeric silsesquioxane to said siloxysilicate comprises from about 1:3 to about 9:11, the combined amount of said iso-octyl polyhedral oligomeric silsesquioxane and said siloxysilicate comprising from about 0.5% to about 35% by weight of said cosmetic composition;
a solvent; and
a coloring agent.

22. A method of improving wear and/or pliability of a cosmetic composition, comprising applying the cosmetic composition of claim 9 to skin, hair, or nails.

23. The method of claim 22, wherein said skin is selected from the group consisting of lips, eyelids, and cheeks.
